(19) United States
(12) Patent Application Publication
(10) Pub. No.: US 2008/0021415 A1
(43) Pub. Date: Jan. 24, 2008

(54) Device suitable for connection to a substantially tubular element

(76) Inventors: Anthony Durkin, County Galway (IE); Desmond John Regan, County Galway (IE)

Correspondence Address: JACOBSON HOLMAN PLLC, 400 SEVENTH STREET N.W., SUITE 600, WASHINGTON, DC, 20004, US

(21) Appl. No.: 11/730,808

(22) Filed: Apr. 4, 2007

Related U.S. Application Data
(60) Provisional application No. 60/789,897, filed on Apr. 7, 2006.
(60) Provisional application No. 60/808,946, filed on May 30, 2006.

(30) Foreign Application Priority Data
Apr. 7, 2006 (IE) IE2006/0268

(51) Int. Cl. A61M 39/04

(52) U.S. Cl. 604/246

(57) ABSTRACT

A barb connector (2) for use in a medical drainage device for draining fluid from a pleural cavity of a patient comprises a gripping portion (10), two cylindrical tubular members (11) extending outwardly from the gripping portion (10), and a plurality of annular protrusions (13) spaced-apart along the external surface of each of the tubular members (11). Each tubular member (11) comprises a plurality of recesses spaced-apart along the external surface of the tubular member (11), and the protrusions (13) are mounted in the recesses. Each protrusion (13) is of a compressible material. Upon insertion of a tubular member (11) into a lumen defined through a drain tube of the medical drainage device, the protrusions (13) engage the internal surface of the drain tube and deform to sealingly engage the internal surface.

This invention relates to a device suitable for connection to a substantially tubular element, to a fluid flow indicator, and to a medical drainage device.

In the human anatomy, between the outer surface of the lungs and the chest wall lies an area known as the pleural cavity. Under normal conditions in a healthy patient, the pleural cavity is a closed space and is at negative pressure or vacuum so that the lungs fully expand during the respiration inhalation. When the chest wall is opened, for example as a result of surgery or chest injury, the in-rush of air causes the vacuum in the patient's pleural cavity to be lost and atmospheric air to enter the pleural cavity. Since the normal vacuum is no longer present, the lungs collapse as they depend upon this vacuum to stay fully expanded up against the chest wall. When air enters or becomes trapped inside the pleural cavity, the lungs cannot fully expand, and the patient experiences difficulty breathing. This condition is known as pneumothorax. This is a frequent occurrence after thoracic and/or cardiac surgeries, and also following chest wall injuries. Often there is a combination of both air and blood present in the pleural cavity, causing similar difficulties breathing.

Generally a physician's prescribed treatment for these clinical situations is to remove the fluid as promptly as possible, to prevent evacuated fluid from returning into the pleural cavity, and to expand the lungs and restore the negative pressure in the pleural cavity to its normal level. To facilitate fluid evacuation post surgically, it is known to insert a medical drainage device into the pleural cavity.

### STATEMENTS OF INVENTION

According to the invention there is provided a device suitable for connection to a substantially tubular element, the device comprising:-
at least one substantially tubular member; and
one or more protrusions on a surface of the at least one tubular member;
the one or more protrusions being at least partially substantially deformable, upon engagement with a substantially tubular element, to sealingly engage the element.

Because the protrusions deform upon engagement with the tubular element, this arrangement results in an enhanced sealing effect between the protrusions and the tubular element.

In one embodiment of the invention the protrusion is at least partially substantially compressible. Preferably the protrusion is radially deformable.

In one case the protrusion is mounted to the tubular member. Preferably the surface of the tubular member comprises a recess into which the protrusion is mounted. By mounting the protrusion in the recess, this provides for a more secure means of mounting the protrusion to the tubular member. Ideally the protrusion is over-moulded to the tubular member.

In one case the protrusion is formed integrally with the tubular member.

In one embodiment the protrusion is provided on an external surface of the tubular member. Preferably the protrusion upstands from the external surface of the tubular member. Ideally the tubular member is insertable at least partially into a substantially tubular element.

In another embodiment the tubular member defines a lumen therethrough. Preferably the protrusion is provided on an internal surface of the tubular member. Ideally a substantially tubular element is insertable at least partially into the tubular member.

In another case the device comprises a plurality of protrusions spaced along the tubular member. Preferably the device comprises a first protrusion and a second protrusion, the second protrusion having a smaller radial dimension than the first protrusion. By having protrusions of different radial dimension, this arrangement accommodates connection of the device to tubular elements of varying size. Ideally the second protrusion is closer to a free end of the tubular member than the first protrusion.

In one embodiment the device comprises a first substantially tubular member and a second substantially tubular member. Preferably the longitudinal axis of the first tubular member is substantially aligned with the longitudinal axis of the second tubular member.

In another case the longitudinal axis of the first tubular member is substantially out of alignment with the longitudinal axis of the second tubular member. The device may comprise a third substantially tubular member. Preferably the device is substantially “Y”-shaped.

In one embodiment the protrusion extends circumferentially around the tubular member. Preferably the protrusion is substantially annular in shape. The tubular member may be substantially cylindrical in shape. In one case the tubular member is substantially rigid.

In one case the protrusion is of a first material and the tubular member is of a second material different to the first material. Preferably the first material is more deformable than the second material. Ideally the first material is more flexible than the second material.

The invention provides in one case a medical device. The invention provides in one case a medical drainage device.

In another aspect of the invention there is provided a medical drainage device comprising a fluid flow indicator, the fluid flow indicator comprising:-
an indicator member defining a lumen therethrough, through which a fluid may flow;
at least part of the indicator member being movable to visually indicate flow of a fluid through the indicator member.

The visual indication provides a simple and fast means of indicating to an operator whether fluid is flowing.

The movable indicator member provides a convenient, dry means of indicating to an operator whether fluid is flowing.

In one embodiment of the invention the indicator member is movable between a flow configuration to visually indicate flow of a fluid through the indicator member, and a rest configuration to visually indicate substantial cessation of flow through the indicator member. Preferably the indicator member is inflatable to visually indicate flow of a fluid through the indicator member. Ideally the indicator member is substantially inflated in the flow configuration. Most preferably the indicator member is substantially deflated in the rest configuration. The indicator member may comprise a balloon member.

In one case the indicator comprises a sampling member defining a lumen therethrough, through which a fluid may flow to facilitate sampling of the fluid. Preferably the sampling member comprises an outlet. Ideally the sampling member comprises a seal member at the outlet. Most preferably the seal member is movable between an open configuration to facilitate passage of a fluid through the outlet, and a sealed configuration. The seal member may be biased towards the sealed configuration. Preferably the seal member is movable from the sealed configuration to the open configuration upon insertion of an element through the seal member. Ideally the sampling member lumen and the indicator member lumen are connectable in fluid communication with a single source of fluid.

In one embodiment the indicator comprises a control member to selectively control flow of a fluid through the indicator member and/or through the sampling member. Preferably the control member comprises a clamp member to selectively obstruct the indicator member lumen and/or the sampling member lumen. Ideally the control member comprises a handle member to facilitate manual operation of the control member. Most preferably the control member is movable between a first configuration and a second configuration. In the first configuration the control member may be configured to facilitate flow of a fluid through the indicator member and to substantially prevent flow of a fluid through the sampling member. In the second configuration the control member may be configured to facilitate flow of a fluid through the sampling member and to substantially prevent flow of a fluid through the indicator member.

In one case the indicator comprises a support member to support the indicator member. Preferably at least part of the support member is located within the interior of the indicator member. Ideally the support member comprises a tube. Most preferably the tube comprises one or more openings for fluid flow between the tube and the indicator member and/or between the tube and the sampling member.

In another embodiment the indicator comprises a housing member to house the indicator member. Preferably the housing member comprises at least one window to facilitate viewing of the indicator member. Ideally at least part of the indicator member is engagable against the housing member.

The indicator member may be configured to visually indicate flow of a fluid from a pleural cavity of a patient. The indicator member may be configured to move from the rest configuration to the flow configuration upon inhalation of a patient. The indicator member may be configured to move from the flow configuration to the rest configuration upon exhalation of a patient.

In a further aspect the invention provides a medical drainage device suitable for connection to a receptacle for draining of a body fluid into the receptacle, the device comprising:
at least one outlet and at least one valve member at the at least one outlet;
the at least one valve member being movable between a venting configuration to facilitate flow of a fluid out of a receptacle through the outlet, and a sealed configuration to substantially prevent flow of a fluid into a receptacle through the outlet.

The valve member enables positive pressure gas/fluid in the receptacle to be vented to the atmosphere, while preventing atmospheric air from entering the receptacle. If atmospheric air were permitted to enter the receptacle, this could lead to fluid passing into the body, for example air leaking into a pleural cavity of a patient.

The medical drainage device of the invention may be employed in a variety of possible medical applications. For example the device may be employed as a chest drain device to drain fluid from a chest region of a patient, such as a pleural cavity, and/or the device may be employed in a urological application as a urine drain device to drain fluid from a bladder region of a patient.

In one embodiment of the invention the valve member is biased towards the sealed configuration.

In one case the device comprises a substantially tubular body portion. Preferably the device defines a fluid drain lumen, through which a fluid may flow towards a receptacle. Ideally the device defines a fluid vent lumen, through which a fluid may flow towards the outlet. Most preferably the fluid vent lumen and the fluid drain lumen are arranged co-axially. The co-axial lumen arrangement prevents gas/fluid coming in contact with the one way valve member during normal drainage and acts as an escape route for gas pressure build up. It also provides a safety release for gas/fluid in a situation where the collection vessel receptacle becomes over filled. At least one of the fluid drain lumen and the fluid vent lumen may be annular in shape. Preferably the fluid vent lumen is arranged around the fluid drain lumen.

In one case the device comprises two or more valve members. Preferably the valve members are evenly spaced around the body portion. Ideally the device comprises a first valve member and a second valve member, the first valve member being arranged diametrically opposed to the second valve member.

In another embodiment the device comprises a shield member to shield the valve member. The shield member acts as a form of protection to prevent damage to the valve member. Preferably the shield member at least partially covers the valve member. Ideally the shield member comprises one or more openings to facilitate flow of a fluid from the outlet through the one or more openings.

In one case the device is configured to be connected to a drain tube for draining of a body fluid into the device. Preferably the device comprises an anti-kink member to minimise the possibility of kinking at the connection of the body portion to a drain tube. The anti-kink member acts to minimise the possibility of the drain tube kinking in the region where the drain tube is connected to the body portion. Ideally the anti-kink member comprises a sleeve member. Most preferably the sleeve member extends at least partially along the body portion. The sleeve member may be configured to extend at least partially along a drain tube. Preferably the sleeve member is overmoulded or bonded to the body portion and/or is configured to be overmoulded or bonded to a drain tube.

In another embodiment the device is configured to be connected to a receptacle in a sealed manner. Preferably the device is configured to be connected to a receptacle in a snap-fit manner. The snap-fit connection provides a simple, reliable means of assuring an operator that the device is securely connected to the receptacle. Ideally the device is configured to be releasably connected to a receptacle.

In another case the device comprises a receptacle into which a body fluid may be drained. Preferably the receptacle comprises an inlet. Ideally the receptacle comprises a closure member to selectively close the inlet. Most preferably the receptacle comprises a seal member at the inlet. The seal member may be movable between an open configuration to facilitate passage of a body fluid through the inlet, and a sealed configuration. Preferably the seal member is biased towards the sealed configuration. Ideally the seal member is movable from the sealed configuration to the open configuration upon insertion of an element through the seal member. The body portion may be insertable at least partially through the seal member to move the seal member from the sealed configuration to the open configuration.

In one embodiment the receptacle comprises a fluid passageway extending from the inlet. Preferably the fluid passageway extends to a region adjacent to a base of the receptacle. Ideally the fluid passageway comprises a tube.

In one case the receptacle is configured to be carried by a patient. Preferably the device comprises a carrier member, configured to be worn by a patient, for carrying the receptacle. The receptacle may be attached to the carrier member by any suitable means, such as a clip, and/or a belt attachment, and/or a hook-and pile fastener. The carrier member may be provided in any suitable form, such as a strap to be worn over one shoulder and across the torso of the patient. In another case the device comprises a support member for supporting the receptacle. Preferably the support member comprises a support arm movable between a storage configuration and a supporting configuration. Ideally the receptacle is configured to hang from the carrier member and/or from the support member. Most preferably the receptacle is configured to hang substantially upright. By hanging the receptacle upright, this arrangement minimises the possibility of accidental spillage from the receptacle. By always maintaining the collection vessel receptacle vertically upright using the pivot connection arrangement of the belt attachment to the collection vessel receptacle, this ensures that the collection vessel receptacle volume may be easily read using the graduations. Preferably the receptacle comprises a vacuum port. Ideally the device comprises a valve at the vacuum port. The receptacle may comprise a solidifier to solidify a body fluid drained into the receptacle. At least part of the outer surface of the receptacle may be substantially concave in shape. The concave outer surface of the receptacle may conform to the outer surface of a patient to enable the receptacle to be carried/worn in a compact, low-profile manner.

The device of the invention may be configured as a portable/mobile device for use in ambulatory applications. In this case the receptacle into which the body fluid is drained may be mobile. Alternatively the device of the invention may be configured as a stationary/immobile device for use with bed-bound patients. In this case the receptacle into which the body fluid is drained may be stationary.

The volume of the receptacle into which the body fluid is drained may be chosen to suit the particular requirements of a patient. For example the volume of the receptacle may be approximately 30 mL in the case of an ambulatory patient with a persistent small gas/air leak, or the volume of the receptacle may be approximately 500 mL in the case of an ambulatory patient with a medium gas/fluid leak, or the volume of the receptacle may be approximately 2 L in the case of a bed-bound patient with a large gas/fluid leak.

### BRIEF DESCRIPTION OF THE DRAWINGS

The invention will be more clearly understood from the following description of some embodiments thereof, given by way of example only, with reference to the accompanying drawings, in which:-

FIG. 1 is an elevation view of a medical drainage device according to the invention;

FIG. 2 is an isometric view of a barb connector part of the device of FIG. 1;

FIG. 3 is an elevation view of the barb connector part of FIG. 2;

FIG. 4 is an enlarged, elevation view of an end of the barb connector part of FIG. 2;

FIG. 5 is an exploded, isometric view of a fluid flow indicator part of the device of FIG. 1;

FIG. 6 is an elevation view of the fluid flow indicator part of FIG. 5;

FIG. 7 is an end view of the fluid flow indicator part of FIG. 5;

FIG. 8 is an isometric view of a chest drainage unit (CDU) connector part of the device of FIG. 1;

FIG. 9 is an exploded, isometric view of the CDU connector part of FIG. 8;

FIG. 10 is a cut-away, isometric view of the CDU connector part of FIG. 8;

FIG. 11 is a cross-sectional, elevation view of the CDU connector part of FIG. 8;

FIG. 12 is an isometric view of a receptacle of the device of FIG. 1;

FIG. 13 is another isometric view of the receptacle of FIG. 12;

FIG. 14 is an exploded, isometric view of the receptacle of FIG. 12;

FIG. 15 is an isometric view of the device of FIG. 1, in use;

FIG. 15(a) is an isometric view of a support member of the device of FIG. 1 in a storage configuration;

FIG. 15(b) is an isometric view of the support member of FIG. 15(a) in a supporting configuration;

FIG. 15(c) is an isometric view of the support member of FIG. 15(b) and a part of the receptacle of FIG. 12;

FIG. 15(d) is an isometric view of the support member of FIG. 15(b) and the receptacle of FIG. 12;

FIG. 16 is an isometric view of a barb connector part of another medical drainage device according to the invention;

FIGS. 16(a) to 16 (m) are elevation views of barb connectors of other medical drainage devices according to the invention;

FIG. 16(a) is a elevation view of a barb connector of another medical drainage device according to the invention;

FIGS. 16(o) and 16(p) are isometric views of the barb connector of FIG. 16(a);

FIGS. 16(q) to 16(s) are views similar to FIGS. 16(a) to 16(p) of a barb connector of a further medical drainage device according to the invention;

FIG. 17 is an isometric view of an indicator member and a sampling member of a fluid flow indicator part of another medical drainage device according to the invention;

FIG. 18 is an elevation view of the indicator member and the sampling member of FIG. 17;

FIG. 18(a) is an isometric view of an indicator member and a sampling member of a fluid flow indicator part of another medical drainage device according to the invention;

FIG. 18(b) is an exploded, isometric view of the indicator member and the sampling member of FIG. 18(a);

FIGS. 18(c) and 18(d) are views similar to FIGS. 18(a) and 18(b) of an indicator member and a sampling member of a fluid flow indicator part of another medical drainage device according to the invention;

FIG. 18(e) is an elevation view of the indicator member and the sampling member of FIGS. 18(c) and 18(d);

FIG. 19 is an elevation view of a further medical drainage device according to the invention;

FIG. 20 is an elevation view of the device of FIG. 1 and two alternative receptacles;

FIGS. 21 and 22 are isometric views of other medical drainage devices according to the invention, in use;

FIG. 22(a) is an isometric view of a receptacle of another medical drainage device according to the invention;

FIG. 22(b) is an elevation view of the receptacle of FIG. 22(a);

FIG. 22(c) is an exploded, isometric view of part of the receptacle of FIG. 22(b);

FIG. 22(d) is an isometric view of a receptacle of another medical drainage device according to the invention;

FIG. 22(e) is an isometric view of a receptacle of a further medical drainage device according to the invention;

FIG. 23 is an elevation view of a further medical drainage device according to the invention and an alternative receptacle; and

FIG. 24 is an isometric view of a receptacle of a further medical drainage device according to the invention.

### DETAILED DESCRIPTION

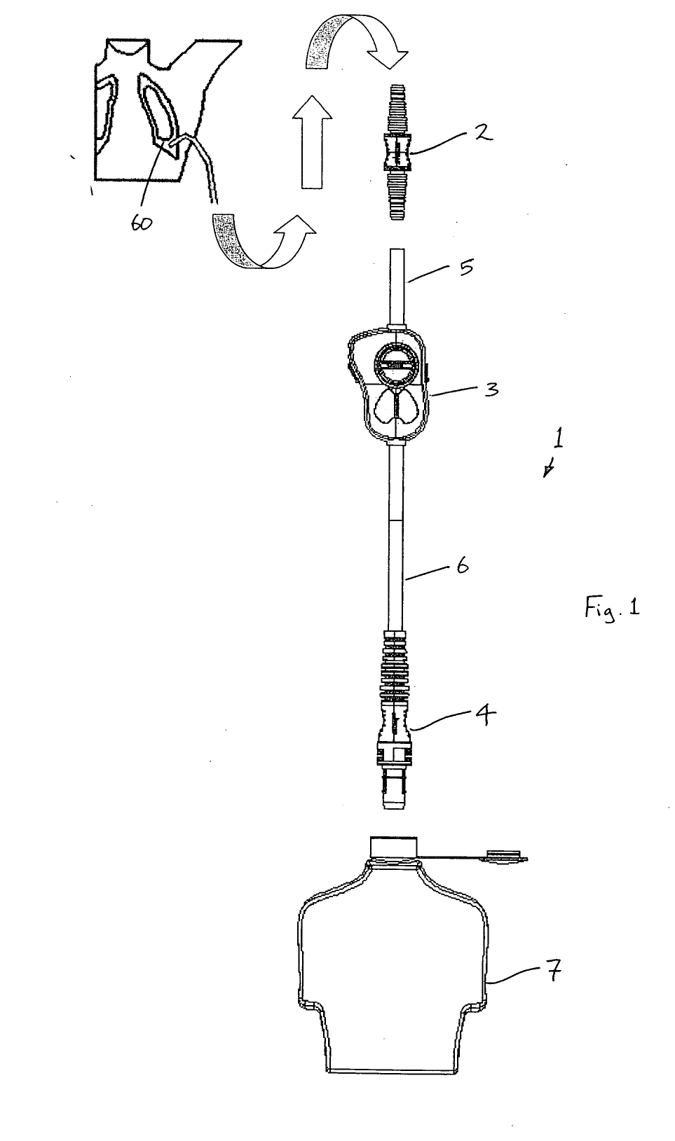

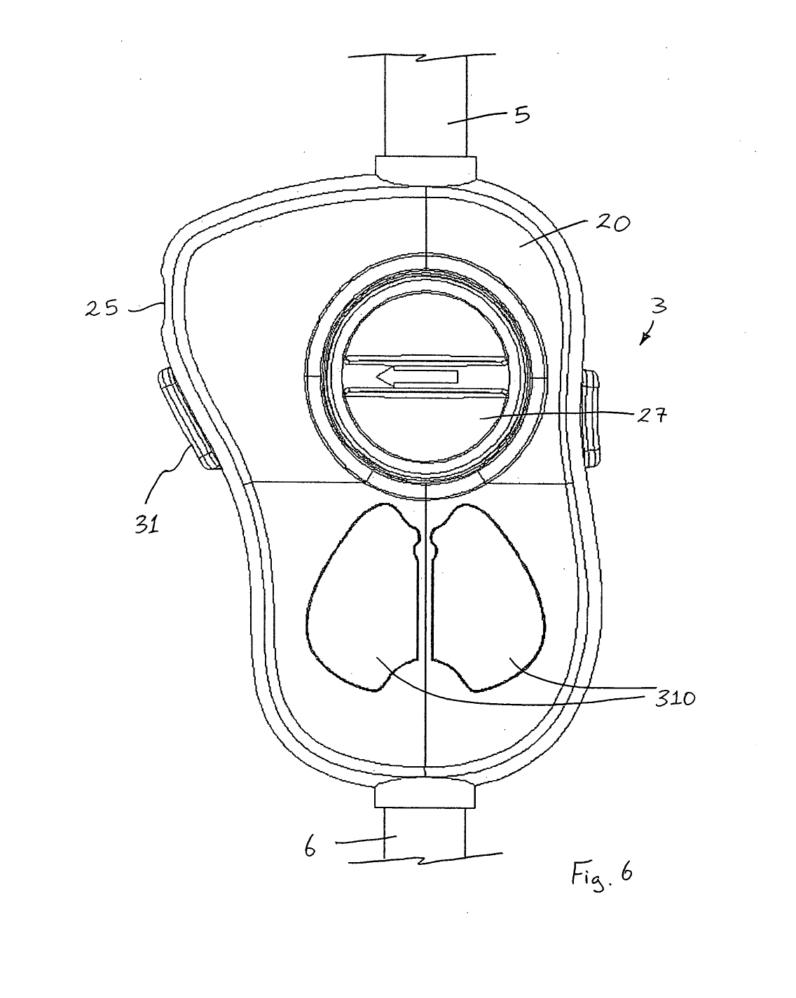

Referring to the drawings, and initially to FIGS. 1 to 15 thereof, there is illustrated a medical drainage device 1 according to the invention. The device 1 is particularly suitable for draining fluid from a pleural cavity 60 of a patient. The fluid may be drained from the pleural cavity 60 by relying on gravitational force only or alternatively suction may be applied to assist in drainage of the fluid.

In this patent specification, the term fluid will be understood to mean a gas, such as air, or a liquid, such as blood, or a combination of a gas and a liquid.

The device 1 comprises a barb connector 2, a fluid flow indicator 3, a chest drainage unit (CDU) connector 4, a tubular upper drain tube 5 between the barb connector 2 and the fluid flow indicator 3, a tubular lower drain tube 6 between the fluid flow indicator 3 and the CDU connector 4, and a receptacle 7.

The barb connector 2 is suitable for being connected to the upper drain tube 5.

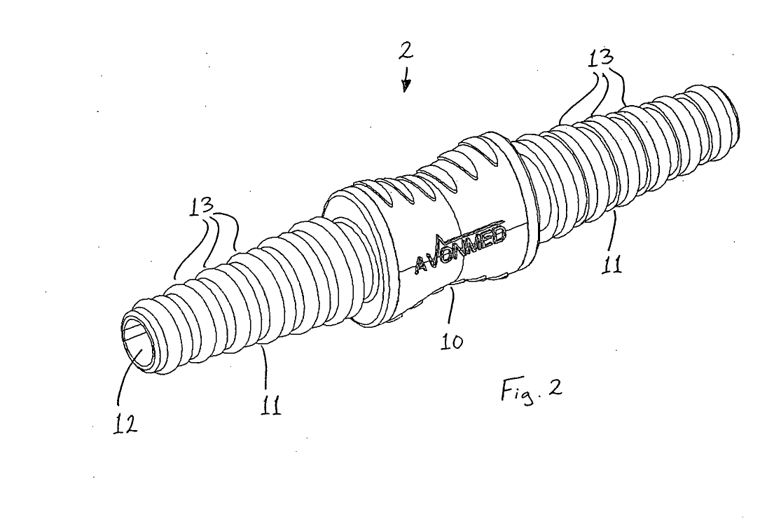

As illustrated in FIGS. 2 to 4, the straight barb connector 2 comprises a gripping portion 10, and two cylindrical tubular members 11 extending outwardly from the gripping portion 10. The longitudinal axes of the tubular members 11 are aligned with one another, and a lumen 12 is defined through the barb connector 2. The gripping portion 10 is of a compressible material, and the two tubular members 11 are of a rigid material. A suitable material for the gripping portion 10 is thermo plastic elastomer (TPE). A suitable material for the tubular member 11 is polypropylene (PP).

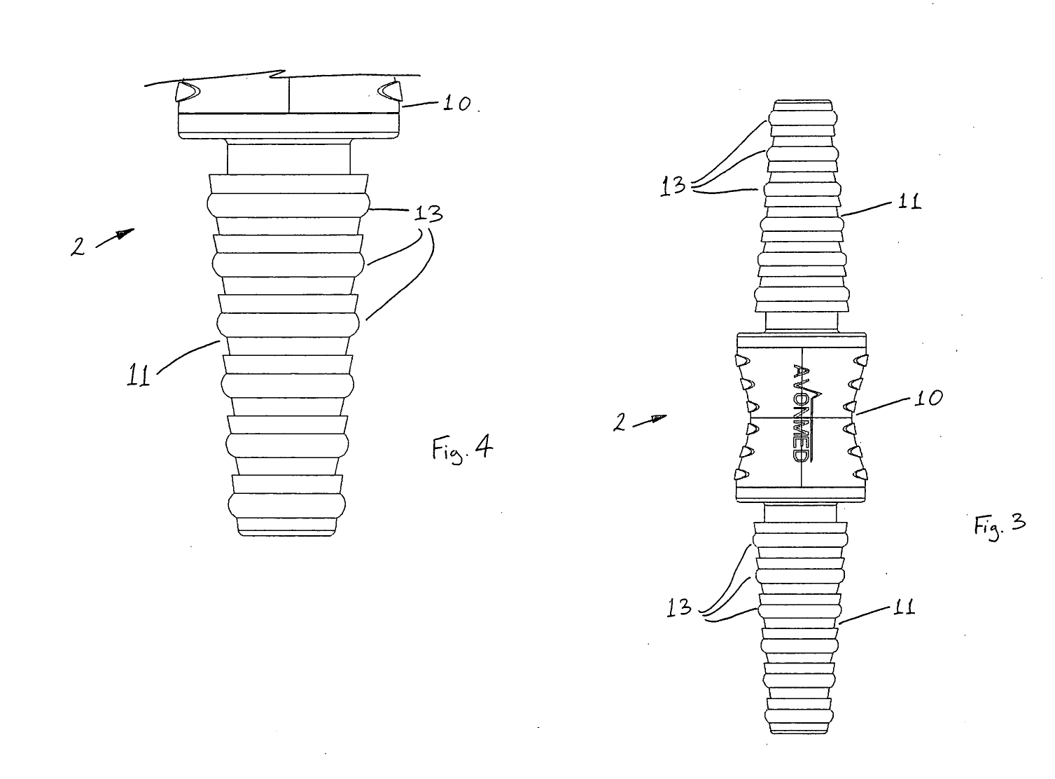

The barb connector 2 comprises a plurality of annular protrusions 13 spaced-apart along the external surface of each of the tubular members 11. Each protrusion 13 extends circumferentially around the tubular member 11. Each tubular member 11 comprises a plurality of recesses spaced-apart along the external surface of the tubular member 11, and the protrusions 13 are mounted in the recesses to mount the protrusions 13 to the tubular member 11, for example by overmoulding the protrusions 13 to the tubular member 11. As illustrated in FIG. 4, the protrusions 13 upstand from the external surface of the tubular members 11.

Each protrusion 13 is of a compressible material and thus is radially deformable. Upon insertion of a tubular member 11 into a lumen defined through the upper drain tube 5, the protrusions 13 engage the internal surface of the upper drain tube 5. Upon engagement with the internal surface, the protrusions 13 deform to sealingly engage the internal surface of the upper drain tube 5. A suitable material for the protrusions 13 is TPE. It will be appreciated however that there are a variety of other suitable medical grade/complaint materials for the protrusions 13.

The material of the protrusions 13 is more deformable and more flexible than the material of the tubular members 11.

As illustrated in FIG. 4, the radial dimension of one protrusion 13 differs from the radial dimension of an adjacent protrusion 13. In particular the radial dimension decreases towards the free end of each tubular member 11. In this manner, the barb connector 2 may be connected in a sealed manner to a variety of differently sized drain tubes.

It will be appreciated that the protrusions 13 may be mounted to the tubular members 11 of the barb connector 2 in a variety of possible manners. For example the protrusions 13 may be formed integrally with the tubular members 11. Alternatively the protrusions 13 may be mounted to the tubular member 11 by, for example, a two-shot moulding process. In an alternative embodiment of the invention, the protrusions 13 may be provided on an internal surface of the tubular members 11. Upon insertion of a tubular element into the barb connector lumen 12, the protrusions 13 may engage the external surface of the tubular element. Upon engagement with the external surface, the protrusions 13 may deform to sealingly engage the external surface of the tubular element.

A catheter or chest tube may be used to drain fluid from the pleural lung space 60 to the barb connector 2.

The function of the barb connector 2 is to provide an air-fluid tight seal when inserted into a catheter, chest tube or any other tubing which requires an air or fluid seal. The body 10, 11 of the connector 2 is a rigid injection moulded part. The series of annular rings 13 are over-moulded on the connector body 11. When the connector 2 is pushed into a catheter, chest tube or any other tube, the annular rings 13 deform to provide an air and fluid tight seal on the internal diameter. The annual rings 13 increase in size along the tubular member 11 to allow a range of internal diameters to be sealed. The moulded annular rings 13 on the barb 2 provide an air and fluid seal when pushed into a catheter or chest tube internal diameter.

The barb connector 2 provides a superior air and fluid sealing method when compared to alternative approaches. Overmoulding of the annular rings 13 provides a unique air/fluid sealing method achieved by combining two dissimilar materials. The barb connector 2 does not rely on rigid sharp edges to provide a seal. This arrangement provides a flexible sealing method. The annular rings 13 can deform to seal non-uniform internal diameters. This arrangement provides low resistance to tube insertion when compared to alternative approaches. This arrangement may result in higher pull forces being required to separate the barb connector 2 from the tube. Thus a tube may be less likely to be accidentally disconnected. The barb connector 2 incorporates the finger grip 10 to ease insertion. The sealing method may be applied to a variety of tube connection applications. It is not limited to use in medical applications.

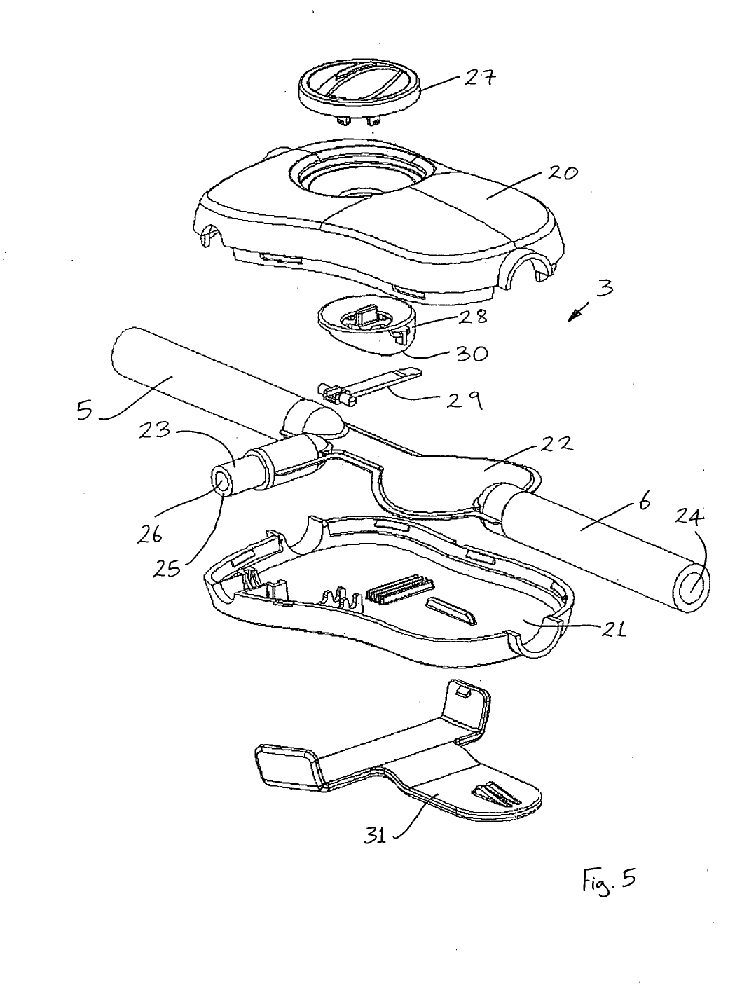

As illustrated in FIGS. 5 to 7, the fluid flow indicator 3 comprises an upper housing part 20, a lower housing part 21, a balloon member 22, a sampling port 23, and a control mechanism to selectively control flow of a body fluid through the balloon member 22 or through the sampling port 23.

The balloon member 22 defines a lumen therethrough which is in fluid communication with the upper drain tube lumen and with a lumen 24 defined through the lower drain tube 6. Upon flow of a body fluid from the upper drain tube 5 into the balloon member 22 and on into the lower drain tube 6, for example upon inhalation of a patient, the balloon member 22 inflates from a deflated rest configuration to an inflated flow configuration. This inflation of the balloon member 22 provides a visual indication to an operator of the medical drainage device I of the flow of the body fluid through the balloon member 22. When flow of the body fluid ceases, for example upon exhalation of a patient, the balloon member 22 deflates from the inflated flow configuration to the deflated rest configuration. This deflation of the balloon member 22 provides a visual indication to the operator of the cessation of the flow of the body fluid through the balloon member 22.

The balloon member 22 may be manufactured by any suitable process, for example by seam welding of sheet plastic, or by blow moulding.

For the fluid flow indicator 3, in particular the blowmoulded balloon member 22, an arrangement may be employed to enhance the indicator movement/feedback by including one or more of the following features:
1. Mould the balloon member 22 from a luminous reflective type material
2. Coat the external surfaces of the balloon member 22 with luminous paint or print a pattern.
3. Texture the external surfaces of the balloon member 22 or add features i.e. ribs or dimples.

The sampling port 23 comprises an outlet 25, and has a lumen 26 defined therethrough which is in fluid communication with the upper drain tube lumen. A body fluid may flow from the upper drain tube 5 through the lumen 26 to the outlet 25 to facilitate sampling of the body fluid.

A seal member is provided in the region of the outlet 25. The seal member is movable from a sealed configuration to an open configuration by inserting an element, such as a needle-less syringe, through the seal member into communication with the sampling port lumen 26. In this open configuration body fluid may pass from the sampling port lumen 26 into the inserted element and out through the outlet 25. The seal member is biased towards the sealed configuration, and thus upon withdrawal of an inserted element the seal member moves from the open configuration to the sealed configuration.

A suitable port for use as the sampling port 23 is available from Bespak—http://www.bespak.com/site/ProductInno/medicalcheck-valves.html.

The control mechanism comprises an upper handle part 27, a lower handle part 28 and a clamp member 29.

The upper handle part 27 is located externally of the upper housing part 20 to facilitate manual operation of the control mechanism. The lower handle part 28 is coupled to the upper handle part 27 such that rotation of the upper handle part 27 causes rotation of the lower handle part 28. The lower handle part 28 has an angled cam surface 30 which is engagable with the clamp member 29 to selectively obstruct the balloon member lumen.

In particular the handle parts 27, 28 are rotatable through 90° between a first configuration and a second configuration. In the first configuration, the cam surface 30 of the lower handle part 28 does not engage the clamp member 29, and the clamp member 29 does not obstruct the balloon member lumen, and thus body fluid may flow from the upper drain tube 5 into the balloon member 22. In this first configuration, the cam surface 30 of the lower handle part 28 prevents flow of the body fluid from the upper drain tube 5 into the sampling port lumen 26. In the second configuration, the cam surface 30 of the lower handle part 28 engages the clamp member 29 to push the clamp member 29 down to obstruct the balloon member lumen, and thus body flow is prevented from flowing from the upper drain tube 5 into the balloon member 22. In this second configuration, body fluid may flow from the upper drain tube 5 into the sampling port lumen 26. The fluid flow indicator 3 acts as a pleural valve.

When the upper housing part 20 is assembled to the lower housing part 21, the housing parts 20, 21 house the balloon member 22, the sampling port 23, the lower handle part 28 and the clamp member 29 within. Two windows 310 are provided in the upper housing part 20 to facilitate viewing of the balloon member 22 to enable an operator to determine whether the balloon member 22 is inflated or deflated.

A belt clip 31 is mounted to the lower housing part 21 to facilitate releasable attachment of the fluid flow indicator 3 to a belt or similar piece of clothing of a patient.

FIG. 5 illustrates the valve knob on/off 27, the top housing 20, the valve retainer 28, the valve clamp 29, the indicator balloon 22, the sample port 23, the indicator assembly 3, the bottom housing 21 and the belt clip 31.

The function of the pleural valve 3 is to provide flow control and indication when the system 1 is in use. The top housing 20 is moulded from clear polycarbonate material or an alternative equivalent transparent material. Part of the surface is intended to resemble the shape of the lungs and is highly polished. The prints on these two surfaces are intended to represent the ribcage.

The bottom housing 21 is moulded from clear polycarbonate material or an alternative equivalent transparent material.

The valve knob 27 is injection moulded. Rotating this knob 27 shuts down the flow of air or fluid from the patient and also prevents reflux. The moulded arrow indicates the direction of flow. This arrangement provides positive feedback when rotated to either the “on” position (6 o'clock) or the “off” position (9 o'clock). The valve 27 is rotated to the “off” position for sampling or changing/replacing the CDU 7.

The valve knob retainer 28 is injection moulded, and retains the valve knob 27 through the top housing 20.

The valve clamp 29 is injection moulded. When the valve knob 27 is rotated to the “off” position, the retainer 28 interacts with the valve clamp 29 in a cam like motion to compress the indicator balloon 22 to give an airtight seal.

The balloon indicator 22 is blow-moulded. The purpose of the indicator 22 is to provide a method of visual indication, which signals whether air is being expelled from the patient's pleural cavity 60 during respiration. The indicator 22 has been designed to mimic the lung motion during the respiration cycle. That is as the patient inhales during the inspiration cycle the diaphragm lifts and the lungs fill with air. Any air present in the pleural cavity 60 during inhalation is expelled through the pleural valve 3 via the catheter or chest tube, causing the indicator 22 to expand. When the patient exhales during the expiration cycle, air in the lungs is vented normally through the mouth or nose. During this period there is no airflow to the pleural valve 3 hence the indicator 22 deflates, similar to the lungs.

The sample port 23 is inserted into the blow mould indicator 22 and glued in place. The port 23 is for use with needle-less syringes. The operation sequence for the sampling port 23 is as follows:

(1) Turn control valve knob 27 to “off” position.

(2) Insert closed syringe into the port 23.

(3) Expand the syringe piston to draw fluid into the syringe chamber.

(4) Withdraw the syringe from the port 23 when sample is adequate.

(5) The sample port 23 is self-sealed when syringe is withdrawn.

The drain tubes 55, 56 are inserted into the blow mould indicator 22 and can be bonded, for example by being glued or RF welded together.

The belt clip 31 is injection moulded, and is attached via recesses in the bottom housing 21. The clip 31 is designed to clip over a body strap or belt or pocket.

The indicator 22 provides a unique method of indicating pleural airflow compared with alternative approaches. The indicator 22 movement provides an exact replication of the respiration cycle. The indicator 22 can only be activated internally by air or fluid. Indication is not influenced by external factors. The valve 3 requires practically no maintenance. The valve 3 has a silent operation. The indicator 22 may be coloured to enhance visual motion. The pleural valve 3 may be applied to a variety of applications where flow indication of air/gasses etc. is required. It is not restricted to use in medical applications.

The CDU connector 4 is suitable for connection to the receptacle 7 for drawing of the body fluid from the lower drain tube 6 through the CDU connector 4 into the receptacle 7.

As illustrated in FIGS. 8 to 11, the CDU connector 4 comprises a tubular body portion 40, two valve members 41, a shield member 42, and a sleeve member 43.

The body portion 40 comprises two outlets 44 diametrically opposed to one another. The body portion 40 defines a fluid drain lumen 45 through which the body fluid may flow from the lower drain tube 6 towards the receptacle 7, and an annular fluid vent lumen 46 through which the body fluid may flow from the receptacle 7 towards the outlets 44. As illustrated in FIG. 11, the fluid vent lumen 46 is arranged co-axially around the fluid drain lumen 45.

A valve member 41 is mounted to the body portion 40 at each of the outlets 44. In particular each outlet 44 comprises a series of openings, with the valve member 41 mounted in the central opening of the outlet 44. The valve members 41 are movable between a venting configuration and a sealed configuration. In the venting configuration, the valve members 41 facilitate flow of the body fluid from the fluid vent lumen 46 out through the outlets 44. In the sealed configuration, the valve members 41 prevent flow of a fluid, such as atmospheric air, into the fluid vent lumen 46 through the outlets 44. The valve members 41 are biased towards the sealed configuration.

A suitable valve for use as the one way umbrella valve 41 is available from Minivalve—p/n UM 085.001 SD http://www.minivalve.com/.

When the CDU connector 4 is assembled (FIG. 8), the shield member 42 extends over the valve members 41 to cover the valve members 41. The shield member 42 thus acts as a shield for the valve members 41 to prevent inadvertent damage to the valve members 41. A series of openings 47 are provided in the shield member 42 to facilitate flow of the body fluid from the outlets 44 through the openings 47 to the atmosphere.

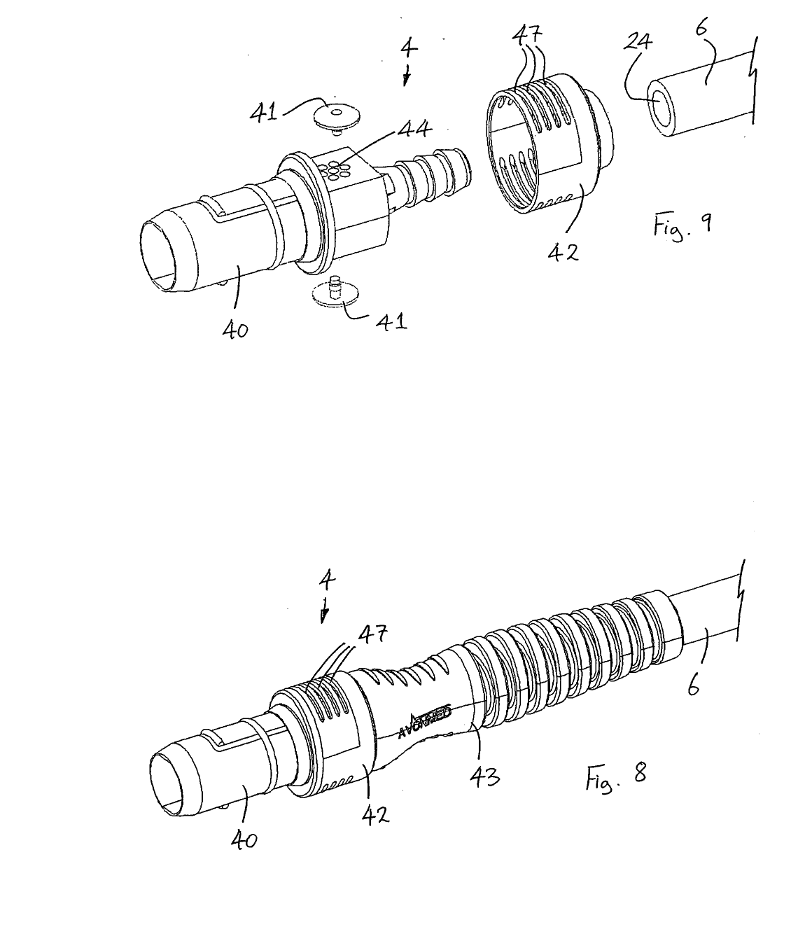

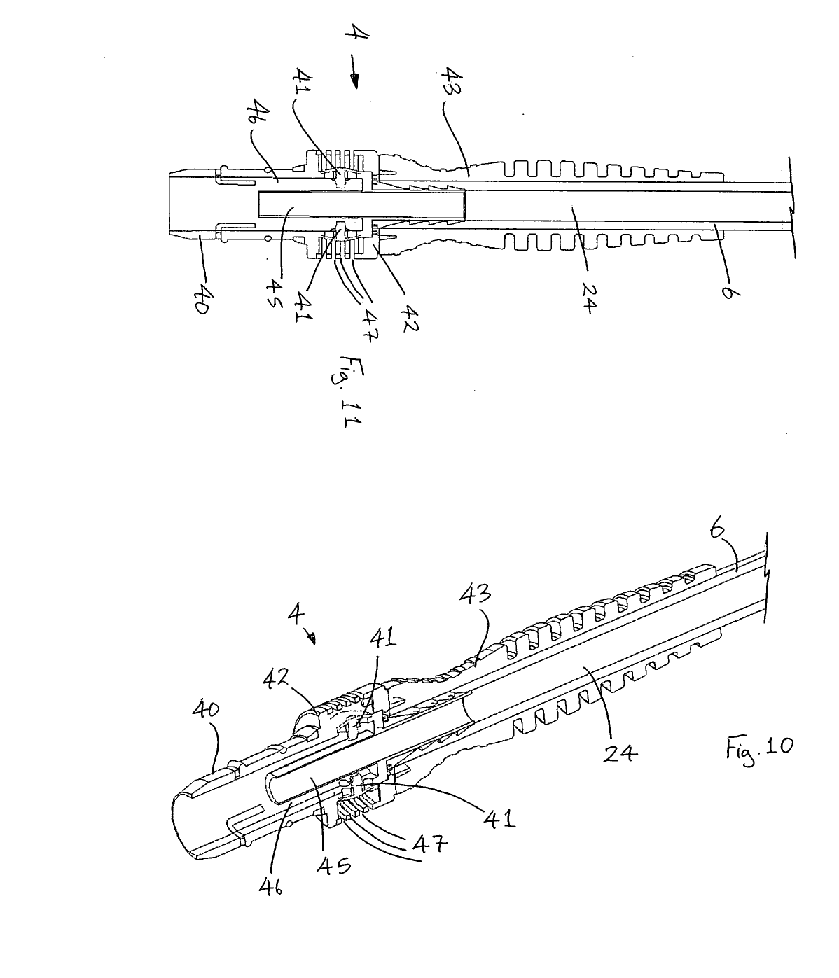

The body portion 40 is connected to the lower drain tube 6 by partially inserting the body portion 40 into the lower drain tube lumen 24 (FIG. 10). When the CDU connector 4 is assembled, the sleeve member 43 extends from the shield member 42 proximally along the lower drain tube 6 (FIG. 11). In this manner, the sleeve member 43 acts to minimize the possibility of kinking at the connection of the body portion 40 to the lower drain tube 6. In particular the sleeve member 43 minimises the possibility of the lower drain tube 6 kinking. The sleeve member 43 may be mounted over the lower drain tube 6 in any suitable manner, for example by overmoulding or bonding.

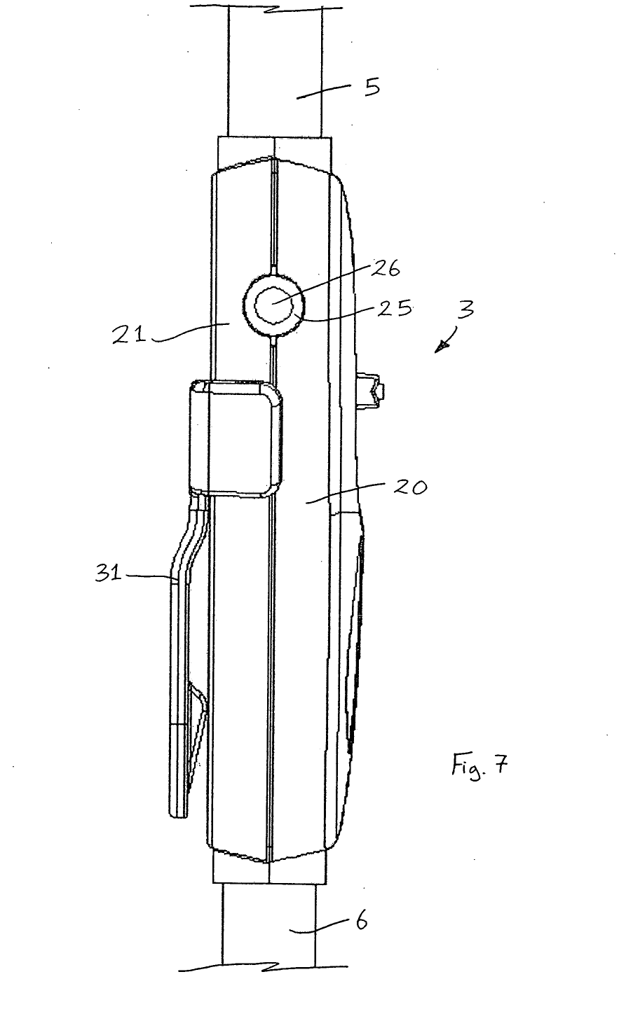

FIG. 9 illustrates the quick connector body 40, the check valves 41, the drain tube 6, the anti-kink tubing 43, the check valve cover 42 and the quick connector overmould 43. When the CDU connector 4 is assembled the sleeve member 43 is overmoulded (FIG. 8).

The function of the CDU connector system 4 is to connect the drain tube 6 from the pleural valve 3 to the CDU 7 and allow the air expelled from the pleural cavity 60 to be vented to the atmosphere. The CDU connector system 4 contains the following parts.

The quick connector body 40 is an injection-moulded part, and is retained in the CDU cap 51 by means of two flexible retaining ribs. The CDU connection/disconnection is achieved by applying a push/pull force to the quick connector system 4.

The check valves 41 have a dual role in the system operation. They crack open under low pressure to allow air in the CDU 7 to be vented to atmosphere. They prevent reflux of air re-entering the system from the atmosphere.

The check valve cover 42 is injection moulded. Its function is to provide protection for the check valves 41.

The drain tube 6 is inserted over a barb on the quick connector body 40.

The quick connector overmould 43 is overmoulded over the CDU connector 4 after assembly. The overmould 43 seals the CDU connector system, provides an ‘O’ ring seal and a flexible/seal on the retaining ribs. It also provides an anti-kink function where the drain tube 6 connects to the quick connector 4.

The CDU connector 4 is a positive action quick disconnection system. The CDU connector 4 may be employed to connect multiple systems. The CDU connector 4 allows automatic venting of CDU air pressure. The CDU connector 4 contains a seal, which prevents air and fluid leaks. The sleeve 43 provides an anti-kink function for the drain tubing 6.

As illustrated in FIGS. 12 to 15, the receptacle 7 comprises an inlet 50, a cap 51 for selective closure of the inlet 50, and a seal member 52 mounted at the inlet 50.

The CDU connector 4 may be releasably connected to the receptacle 7 in a snap-fit manner by partially inserting the body portion 40 of the CDU connector 4 through the seal member 52. The seal member 52 is movable from a sealed configuration to an open configuration by partially inserting the body portion 40 of the CDU connector 4 through the seal member 52. In this open configuration, the body fluid may flow from the CDU connector 4 through the inlet 50 into the receptacle 7. In this open configuration, the seal member 52 seals around the body portion 40 of the CDU connector 4 to ensure that the CDU connector 4 is connected to the receptacle 7 in a sealed manner. The seal member 52 is biased towards the sealed configuration. Thus, upon withdrawal of the body portion 40 of the CDU connector 4 from the seal member 52, the seal member 52 moves from the open configuration to the sealed configuration.

The assembly of the CDU connector 4 to the receptacle 7 provides a closed loop leak proof system. The build up of positive pressure in the collector vessel 7 is relieved to atmosphere via lumen body ports 44 and the check valves 41 being unseated. The check valves 41 prevent the anti-reflux of atmosphere air back into the system by remaining seated on against the lumen body ports 44.

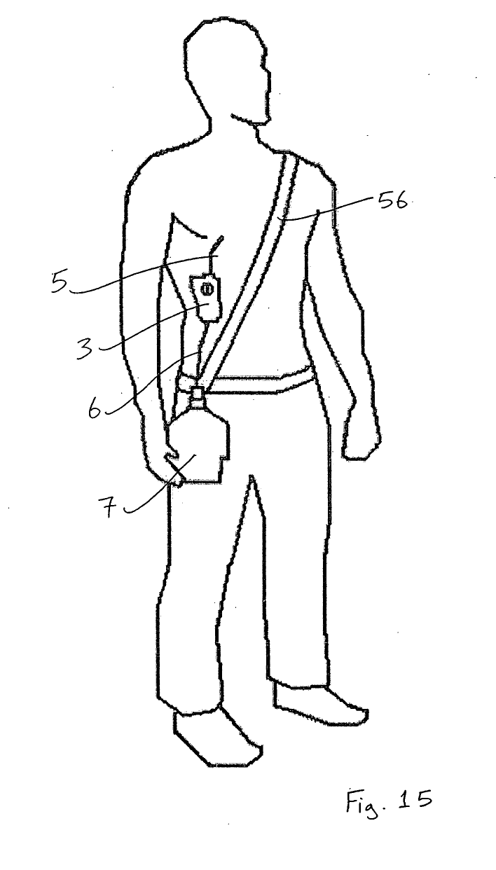

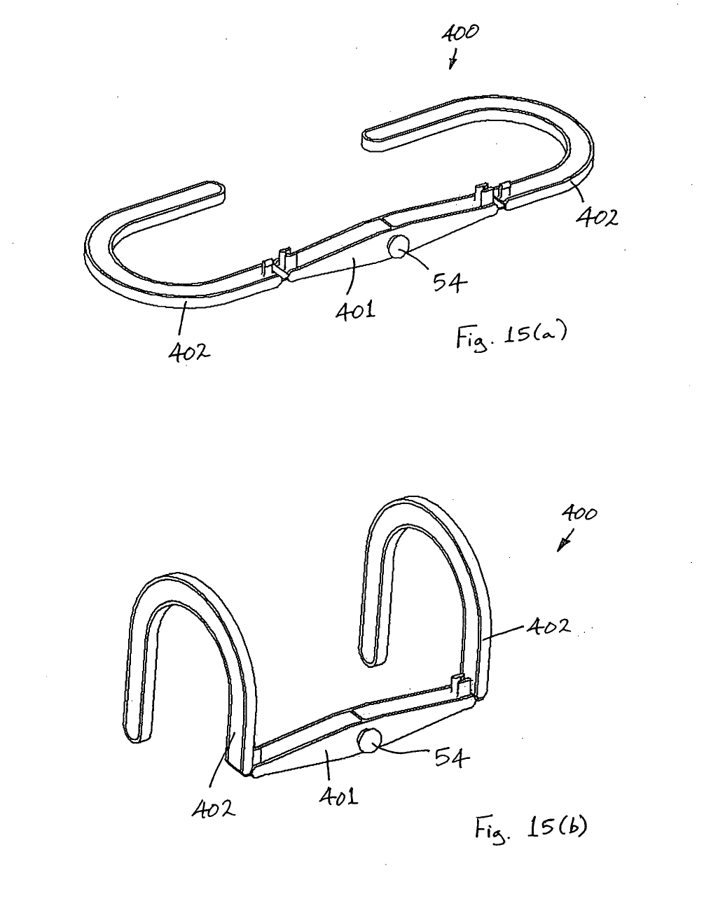

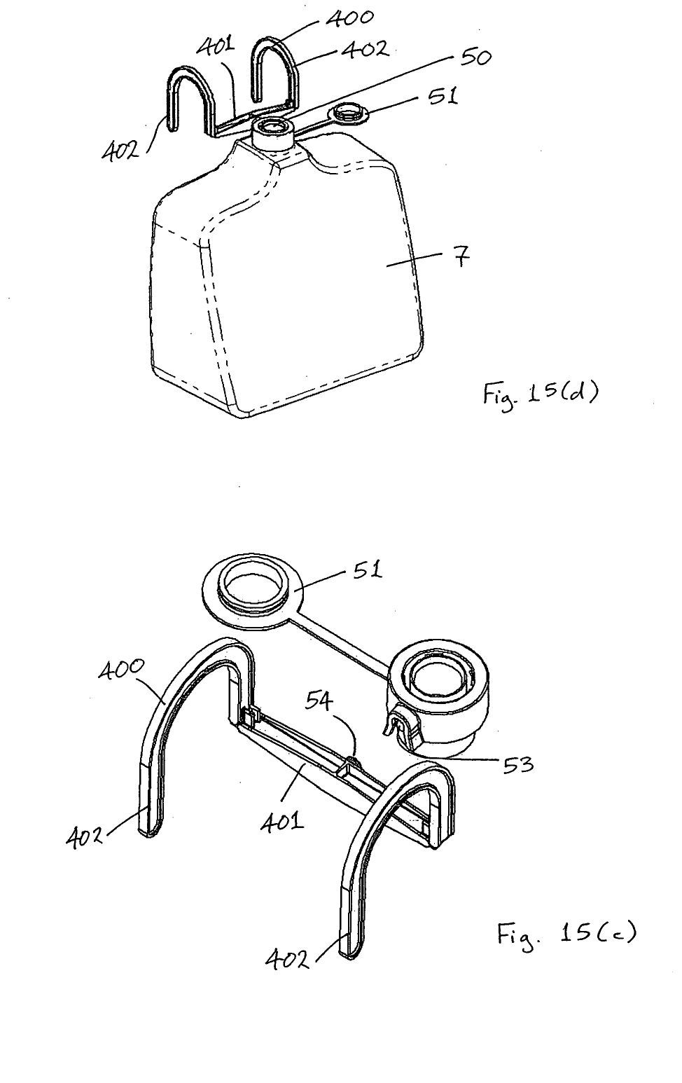

The cap 51 comprises a female recess 53 which is engageable with a corresponding male protrusion 54 on a support element 55 to enable the receptacle 7 to hang from the support element 55 in an upright configuration. In this case the support element 55 is attached to a strap 56 or belt which may be worn by a patient, as illustrated in FIG. 15. The receptacle 7 may therefore be carried hanging from the strap 56. It will be appreciated that the receptacle 7 may be carried in a variety of alternative ways, for example carried in a hand of a patient.

FIGS. 15(a) to 15(d) illustrate an alternative means of supporting the receptacle 7 by means of the support member 400. The support member 400 comprises a main body portion 401 and two curved support arms 402. Each support arm 402 is movable relative to the main body portion 401 between a flat storage configuration (FIG. 15(a)) and a supporting configuration (FIG. 15(b)). The support arms 402 are retained in the supporting configuration by means of a snap-fit engagement with the main body portion 401. The male protrusion 54 is provided on the main body portion 401. In the supporting configuration, the support member 400 may be hung from a suitable element, such as a bed rail. The support member 400 may support the receptacle 7 hanging from the protrusion 54 in an upright configuration.

FIGS. 15(a) and 15(b) illustrate the bed rail hanger 400 in the moulded state (FIG. 15(a)), and in the formed state (FIG. 15(b)). FIG. 15(c) illustrates the support member 400 connecting to the bottle cap. FIG. 15(d) illustrates attached to the bottle 7.

One face 57 of the outer surface of the receptacle 7 is concave in shape. This concave face 57 is arranged to face towards the patient when the receptacle 7 hangs from the strap 56. In this manner the concave face 57 conforms to the outer surface of the patient's leg/torso for a compact, low-profile arrangement.

To minimise the possibility of leakage and/or spillage of the body fluid from the receptacle 7, a solidifier may be provided in the receptacle 7 to solidify the body fluid drained into the receptacle 7.

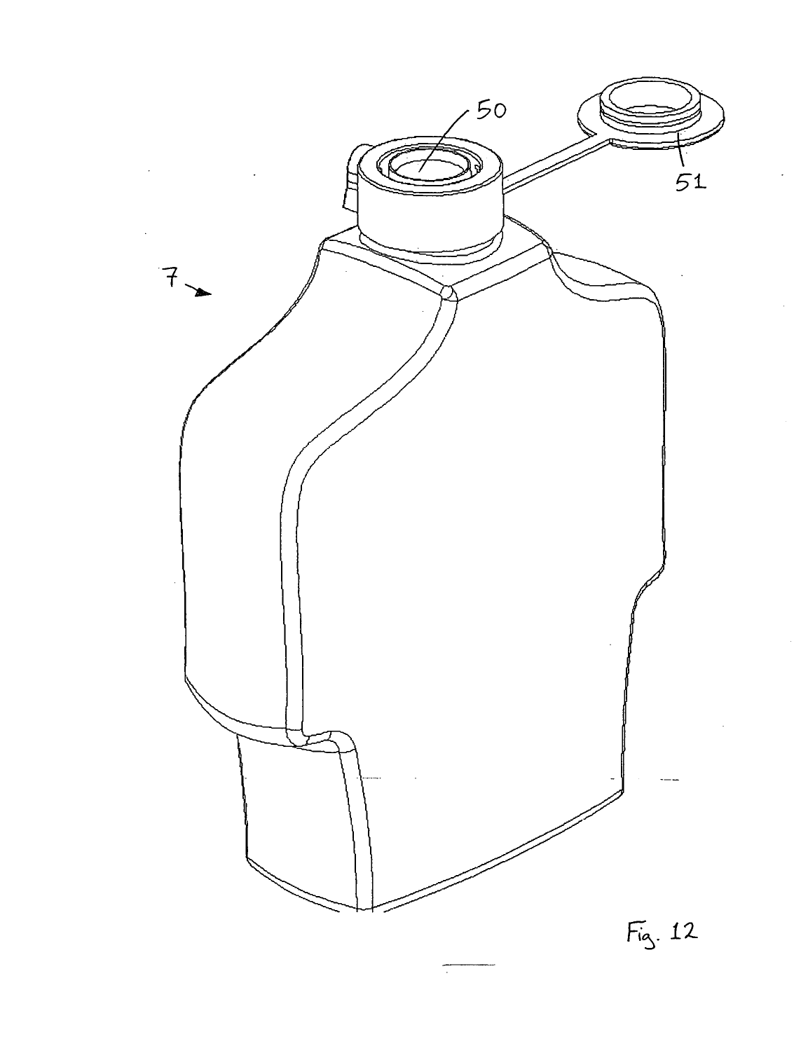

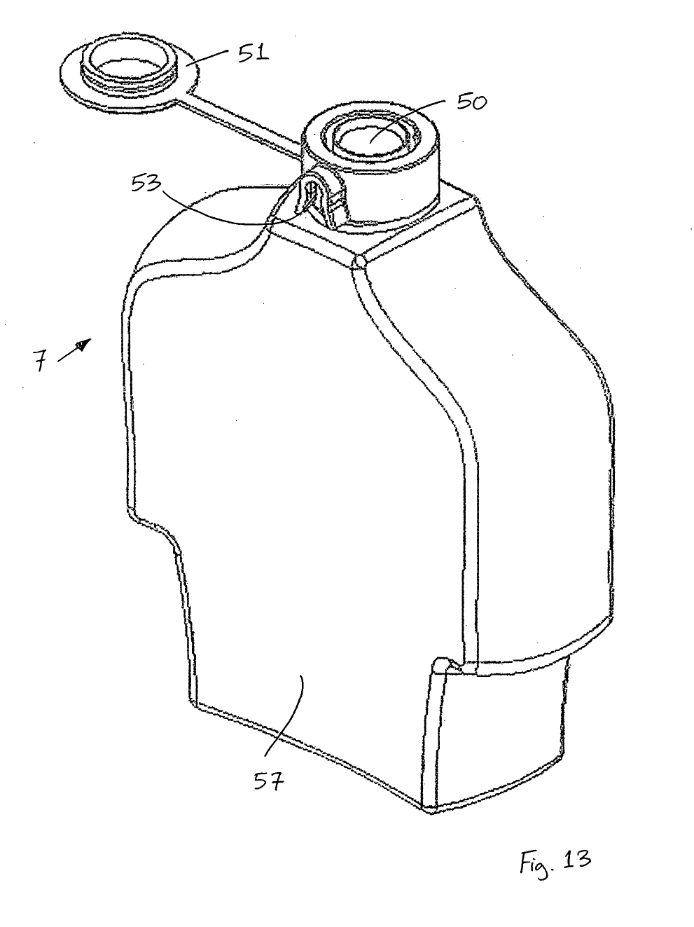

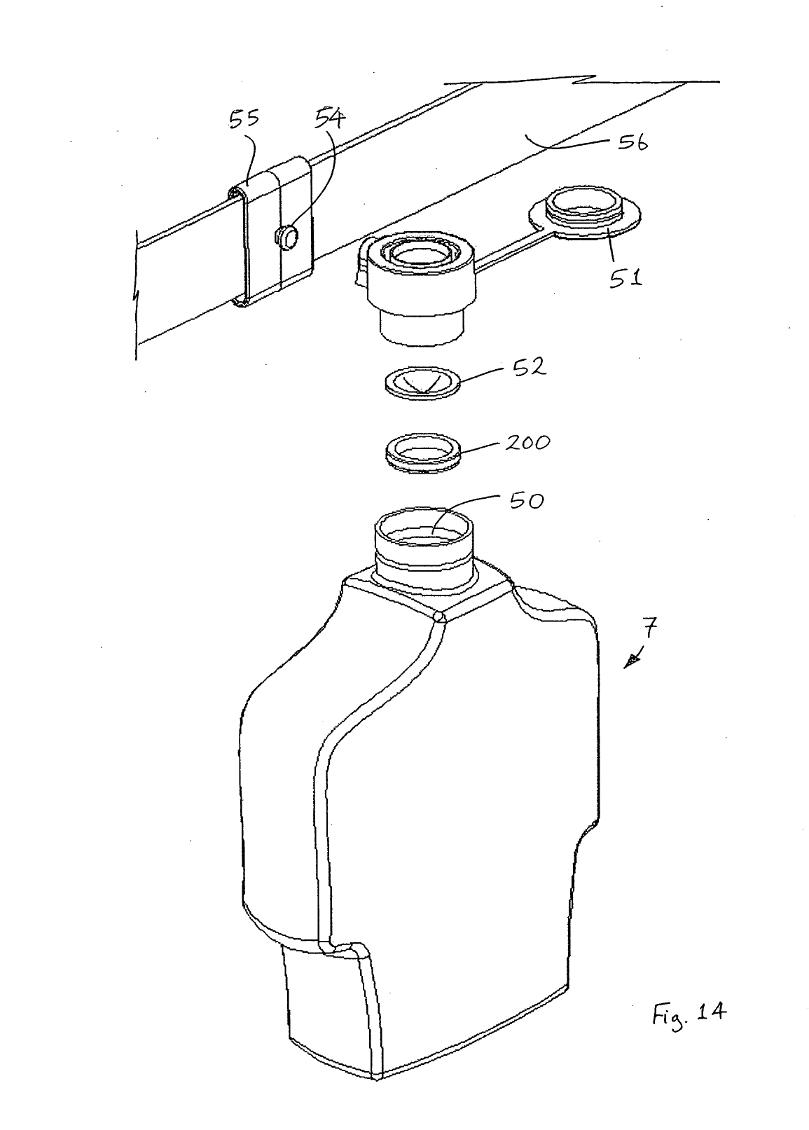

FIG. 14 illustrates the belt strap 56, the belt clip 55, the CDU bottle cap 51, the anti-reflux seal 52, the anti-reflux seal retainer 200, and the CDU 7.

The body strap 56 is used to attach the system 7 to the patient's waist. Hook and loop may provide the closure/adjustment of the belt strap 56.

The body strap attachment 55 is an injection-moulded part. The body strap 56 slides into the belt clip 55. The belt clip 55 can be slid along the length of the belt 56 to provide optimum location. The belt clip 55 has a protrusion to which the CDU bottle cap 51 is retained and pivoted.

The CDU bottle cap 51 is an injection-moulded part. It contains a feature, which enables the CDU assembly 7 to be attached to the belt clip 55.

When the body portion 40 of the CDU connector assembly 4 is inserted into the CDU bottle cap 51 it penetrates the dome shaped seal 52. This penetration opens an ‘X’ cross-slit in the dome seal 52 allowing the free passage of air/fluid into the CDU 7. On withdrawal of the body portion 40 from the CDU assembly 7, the dome seal 52 wipes the contact surfaces and reforms the closed cross-slit ‘X’ seal. The closed cross-slit ‘X’ seal 52 prevent the CDU contents from spilling even if the CDU 7 is inverted or turned upside down.

The retaining ring 200 is used to retain the anti-reflux seal 52 in the CDU bottle cap 51. It snaps into position.

The collection bottle 7 is a blow mould part with printed surfaces. Its function is to act as a collection vessel for the contents as discharged by the patient.

The cap 51 pivots to always hang in the vertical axis. The anti-reflux seal 52 prevents contamination. The CDU may be shaped with a curvature. A solidifier powder may be added into the collector 7. This powder would act as a solidifying agent, which would solidify the drained contents in the collector 7 thus eliminating the possibility of spillage, contamination and would enable safe disposal of drainage contents.

The device 1 is a single use disposable pleural drainage device system. The device 1 may be used in Cardio thoracic medical sector and may be used as an ambulatory or mobile drainage product.

The invention provides a drainage system 1 which connects to a catheter or chest tube, non-invasive application. The proposed system 1 provides a unique method of both controlling and collecting the flow of air and fluid from the patient's pleural space or cavity 60.

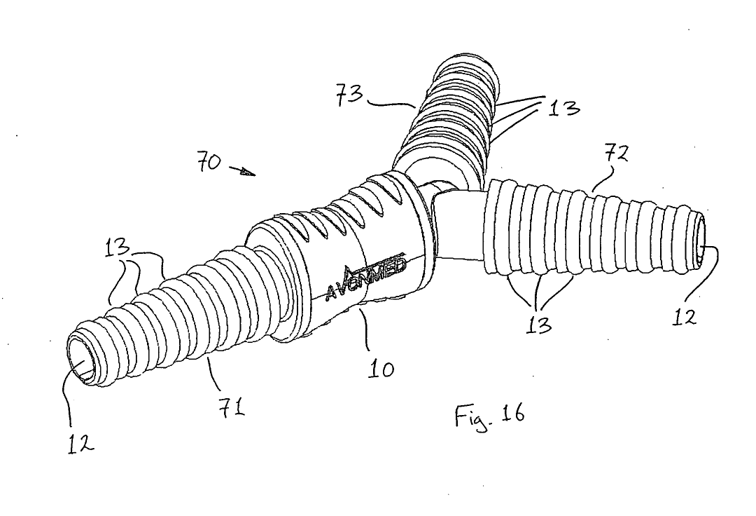

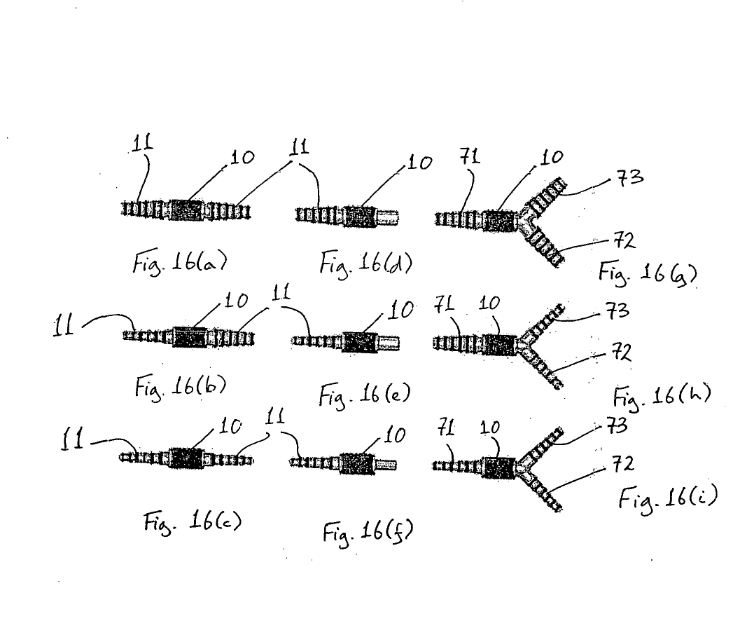

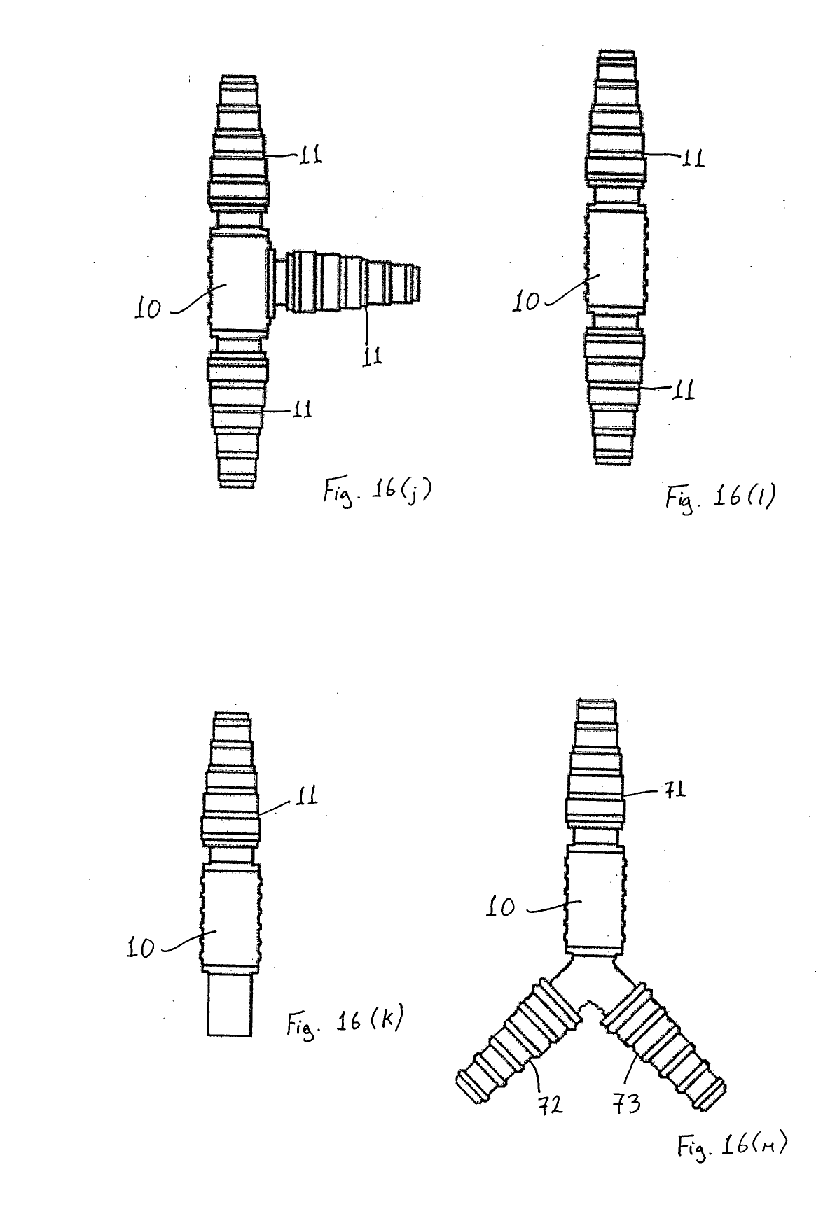

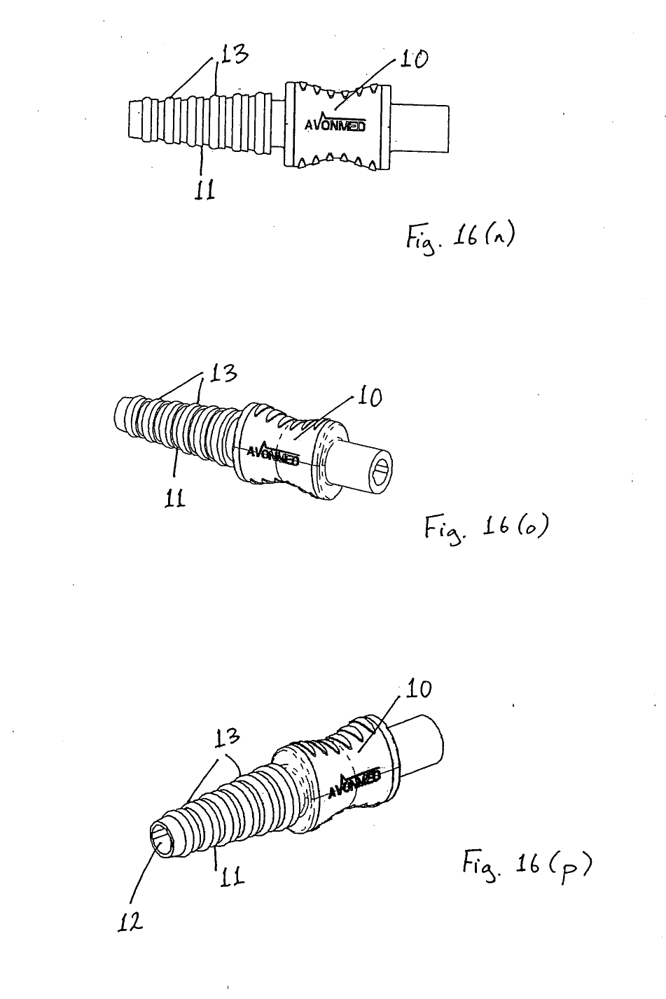

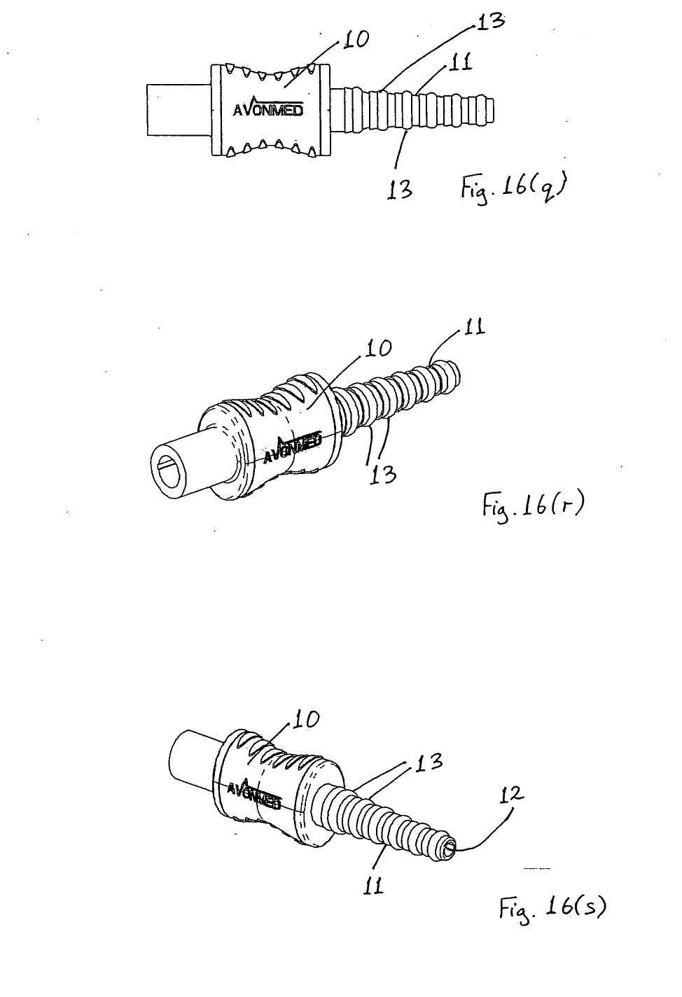

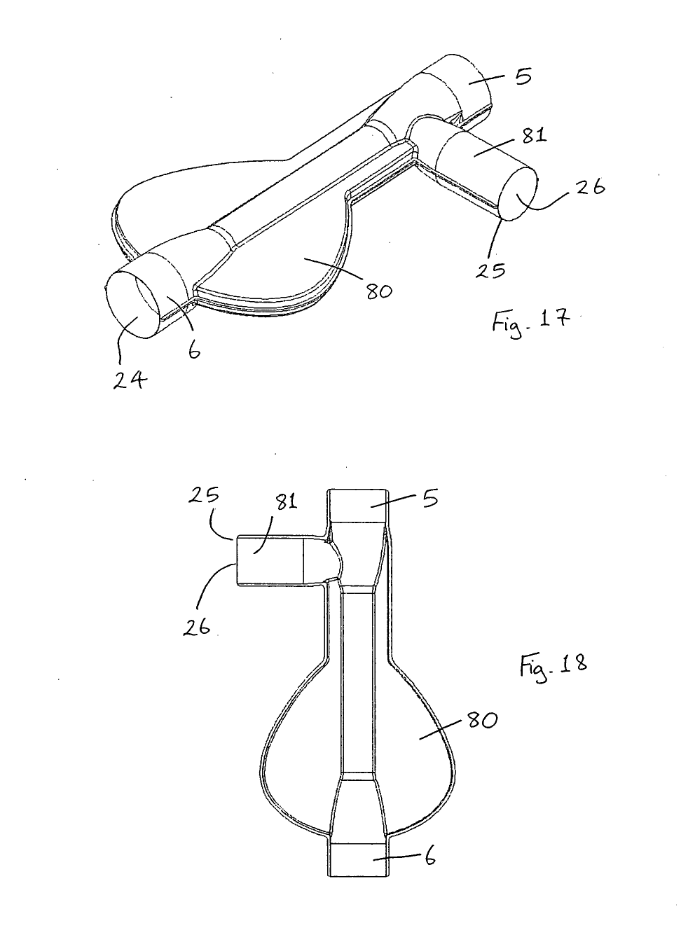

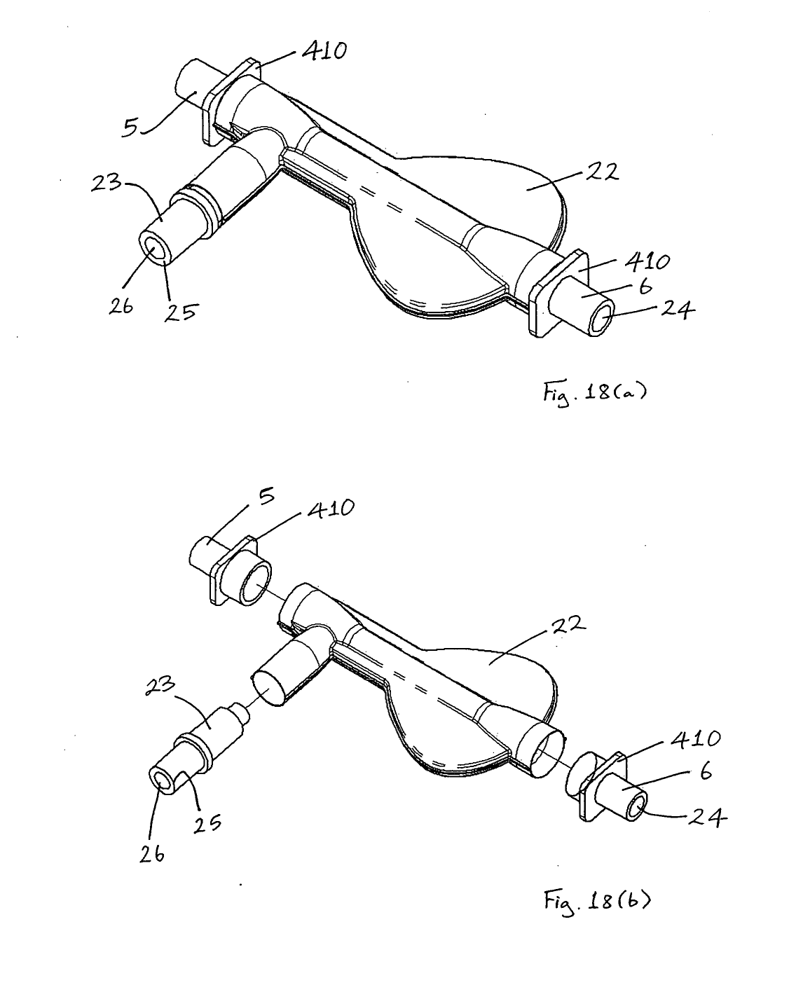

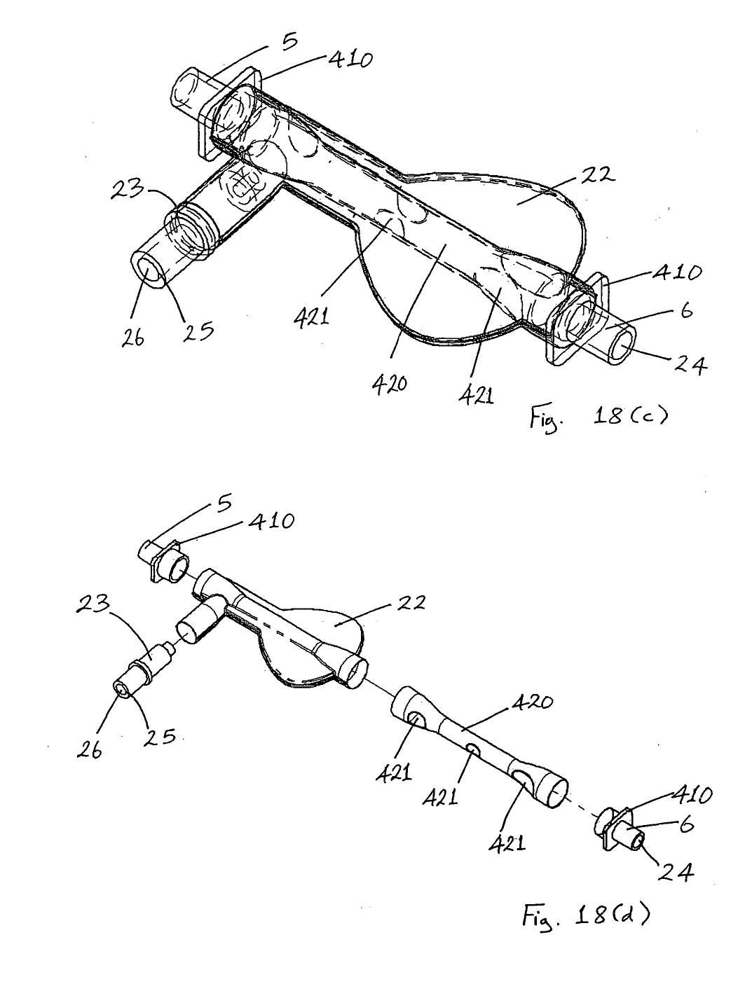

In FIG. 16 there is illustrated a barb connector 70 of another medical drainage device according to the invention, which is similar to the barb connector 2 of FIGS. 2 to 4, and similar elements in FIG. 16 are assigned the same reference numerals.

In this case the barb connector 70 has a “Y-shape”, and comprises three cylindrical tubular members 71, 72, 73. The longitudinal axis of the first tubular member 71 is out of alignment with the longitudinal axes of the second and third tubular members 72, 73. Similarly the longitudinal axis of the second tubular member 72 is out of alignment with the longitudinal axis of the third tubular member 73.

It will be appreciated that the barb connector may be provided in a variety of possible configurations FIGS. 16(a) to 16(s) illustrate various possible configurations for the barb connector.

The barb connector provides a superior grip and seal system, and may be connected to any tube size. It will be appreciated that there could be a variety of catheter connector designs, material options and manufacturing options to make these parts. The profile geometry of the protrusion rings 13 could be arranged differently. The protrusions 13 could be arranged in a spiral instead of individual rings. Some options for the connector design types are shown in FIGS. 16(a) to 16(s).

FIGS. 16(n) to 16(p) illustrate an adult connector size for attachment to a tube on the other end. FIGS. 16(q) to 16(s) illustrate a paediatric connector size for attachment to a tube on the other end.

There are a variety of different possible configurations for the connector . FIGS. 16(a) to 16(s) illustrate a selection of possible configurations.

The connector may be manufactured from two materials. The connector body 11 may be a thermoplastic PP [Polypropylene] and the overmould 13 may be thermoplastic elastomer TPE [Santoprene].

The connector may be manufactured from a variety of combinations of materials. The body 11 may be of any suitably rigid engineering thermoplastic and the overlay material may be of any compressible material, such as TPE or silicone, natural or rubber or other soft.

The connector may be manufactured using any suitable manufacturing process, such as a moulding process, for example a 2-stage process where the body 11 is moulded on one tool and is then transferred to another tool to “overmould” it with the compressible material 13. The connector may also be manufactured using a 2-shot moulding process where all the moulding is done in one tool in the same mould machine. The mould accepts the two materials and is moulded in one process. The stages within the 2-shot process are similar i.e. 2 stages. This may be a partially suitable process for high volume manufacture using multiple cavity tools i.e. moulding multiple parts in one operation.

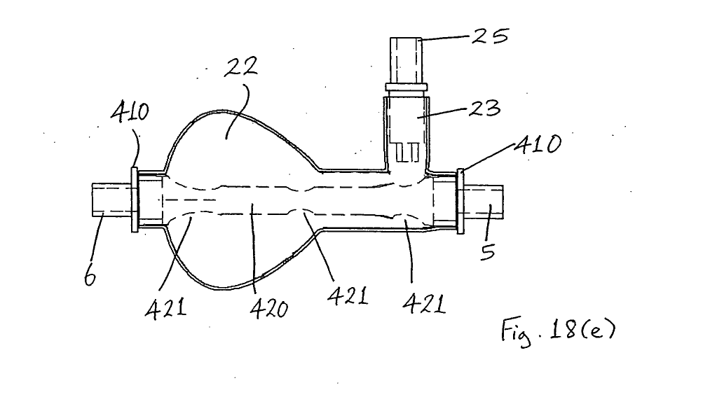

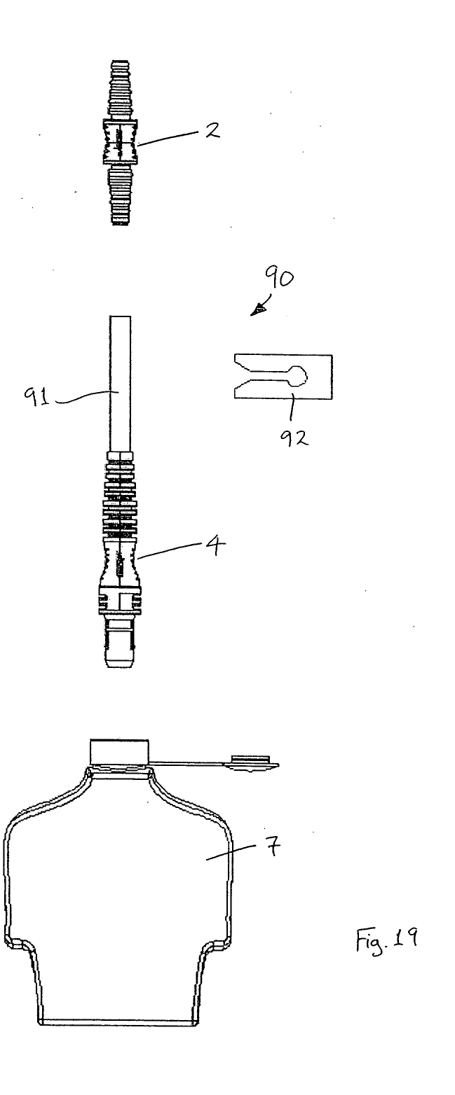

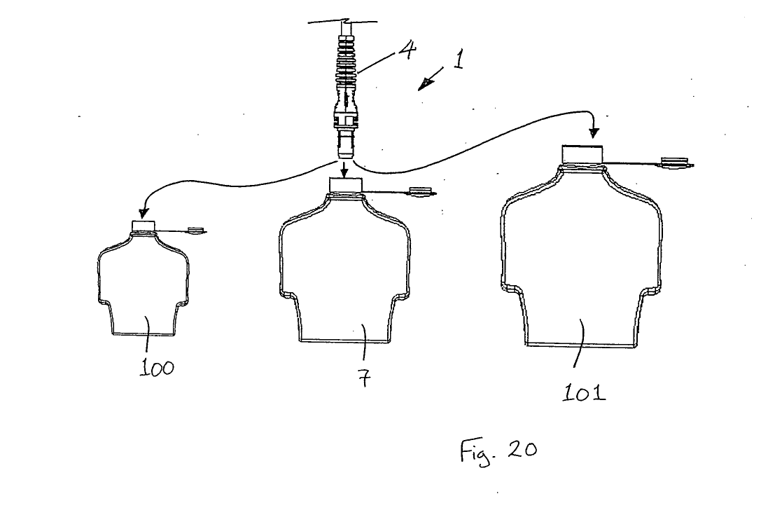

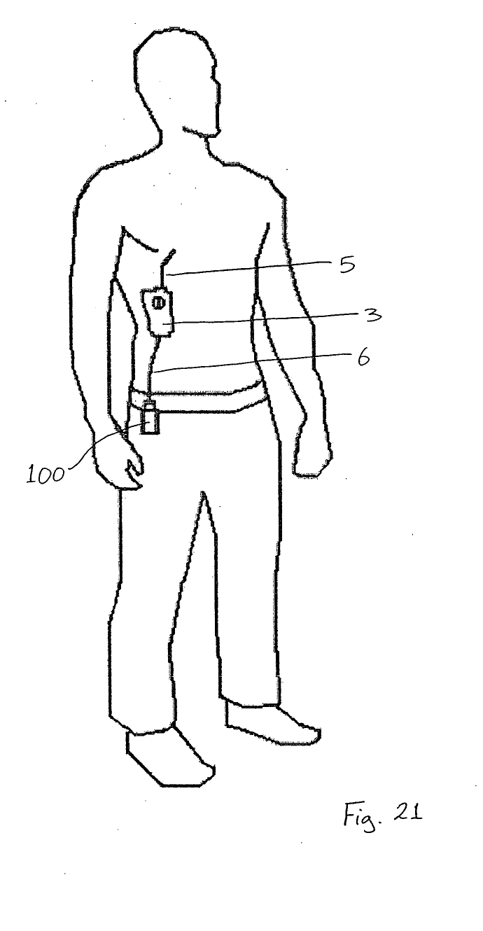

FIGS. 17 and 18 illustrate part of a fluid flow indicator of another medical drainage device according to the invention, which is similar to the fluid flow indicator 3 of FIGS. 5 to 7, and similar elements in FIGS. 17 and 18 are assigned the same reference numerals.

In this case the balloon member 80 and the sampling port 81 are integrally formed, for example by blow moulding. Manufacturing the balloon member 80 by blow moulding may result in enhanced shape and function of the balloon member 80. Blow moulding may also lead to ease of manufacture.

In FIGS. 18(a) and 18(b) there is illustrated part of a fluid flow indicator of another medical drainage device according to the invention, which is similar to the fluid flow indicator 3 of FIGS. 5 to 7, and similar elements in FIGS. 18(a) and 18(b) are assigned the same reference numbers.

In this case the fluid flow indicator comprises a first end plate 410 between the upper drain tube 5 and the balloon member 22, and a seal end plate 410 between the balloon member 22 and the lower drain tube 6. Each end plate 410 is rectangular in cross-section. The end plates 410 engage against the inner surface of the housing parts 20, 21. This engagement prevents any twisting of the drain tubes 5, 6 causing twisting of the balloon member 22.

FIGS. 18(a) and 18(b) illustrate the balloon assembly assembled (FIG. 18(a)), the balloon assembly exploded (FIG. 18(B)), and the balloon connectors 410.

FIGS. 18(c) to 18(e) illustrate part of a fluid flow indicator of another medical drainage device according to the invention, which is similar to the part of the fluid flow indicator of FIGS. 18(a) and 18(b), and similar elements in FIGS. 18(c) to 18(e) are assigned the same reference numbers.

In this case the fluid flow indicator comprises a support tube 420 within the interior of the balloon member 22 extending from the upper drain tube 5 to the lower drain tube 6. The tube 420 has a number of openings 421 for fluid flow between the tube 420 and the balloon member 22 and between the tube 420 and the sampling port 23.

The tube 420 supports the balloon member 22. In the case where a vacuum is applied to the balloon member 22, the tube 420 provides the balloon member 22 with internal strength to prevent damage to the balloon member 22.

The fluid flow indicator of FIGS. 18(c) to 8(e) allows for attachment of vacuum. FIG. 18 (c) illustrates the vacuum balloon assembly: FIG. 18(d) illustrates the vacuum balloon assembly exploded, and the inner tube 420. The eye holes 421 allow sampling of fluid and allow the balloon 22 to expand and contract and fluid to escape. The tube 420 prevents the balloon 22 collapsing when vacuum is applied.

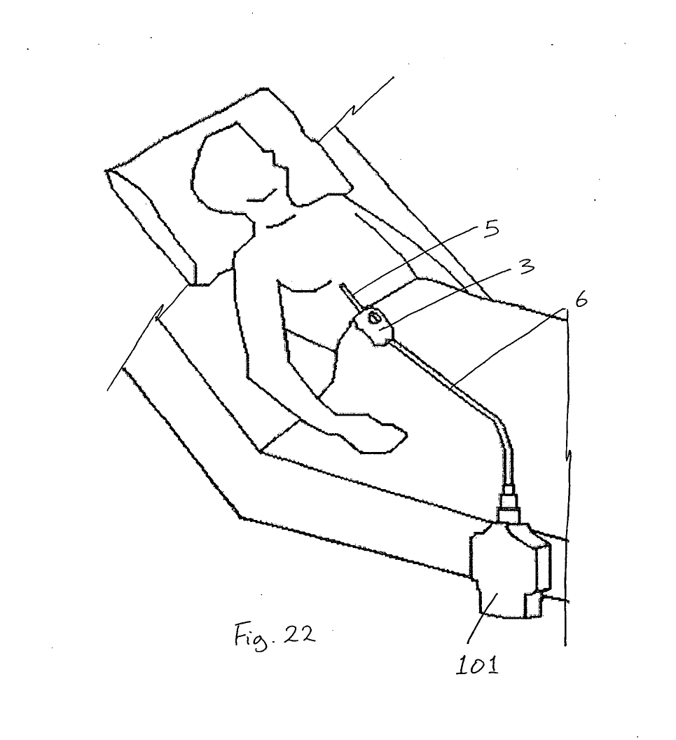

Referring to FIG. 19 there is illustrated a further medical drainage device 90 according to the invention, which is similar to the device 1 of FIGS. 1 to 15, and similar elements in FIG. 19 are assigned the same reference numerals.

In this case a single drain tube 91 connects the barb connector 2 to the CDU connector 4. No fluid flow indicator is provided. A tube slide clamp member 92 may be employed to selectively prevent flow of the body fluid through the drain tube 91 from the barb connector 2 to the CDU connector 4.

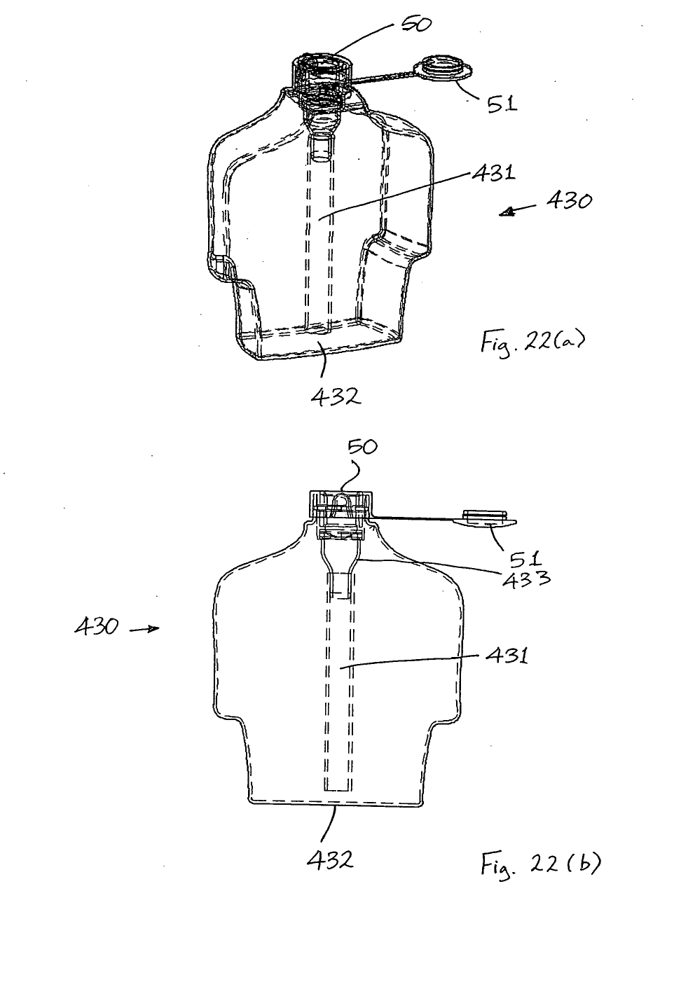

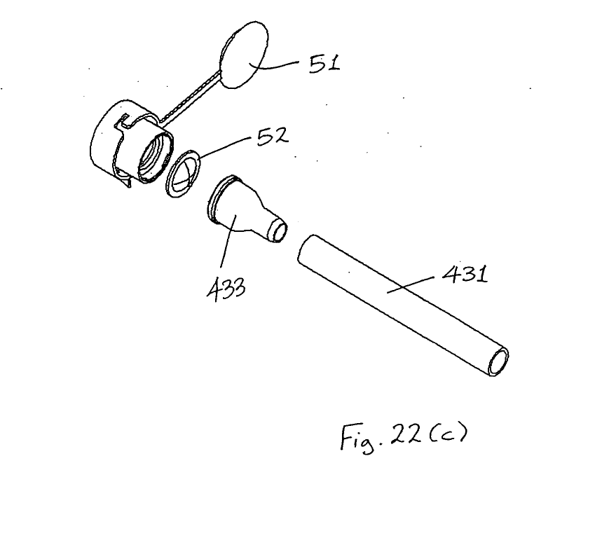

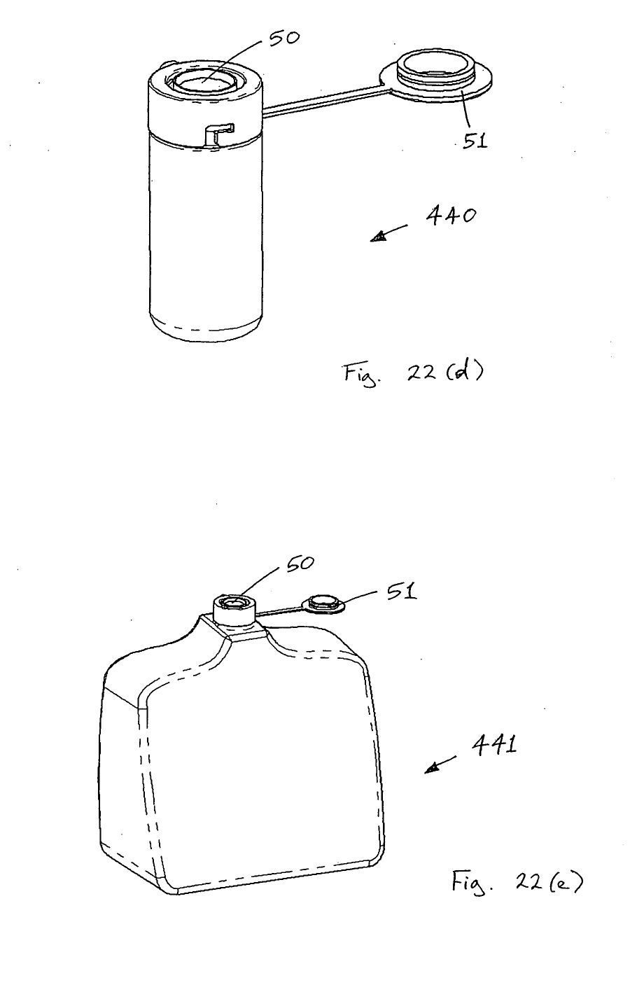

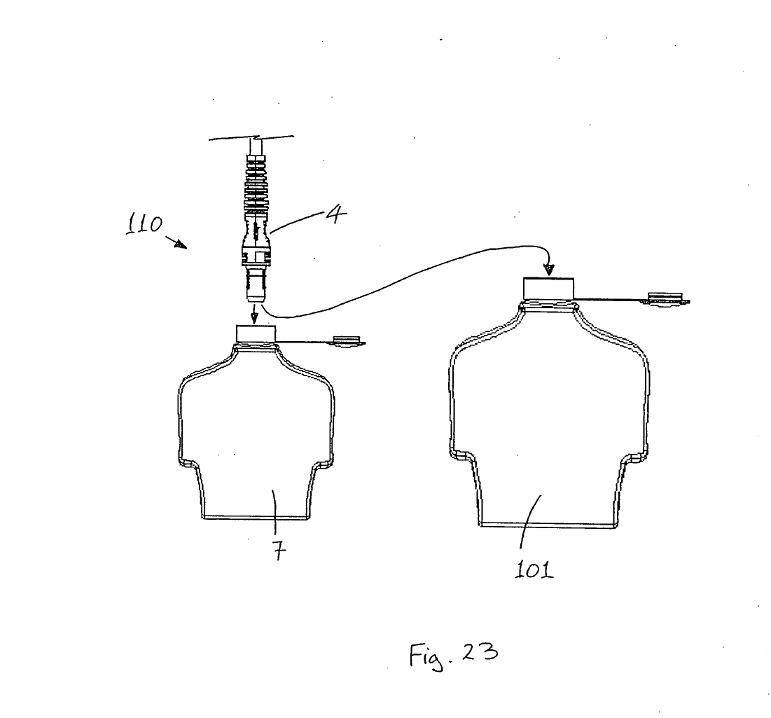

It will be appreciated that the CDU connector 4 is suitable for being connected to a variety of differently sized receptacles (FIG. 20). For example, the CDU connector 4 may be connected to the receptacle 7, as described previously with reference to FIGS. 12 to 15, which may have a volume of approximately 500 mL. Alternatively the CDU connector 4 may be connected to a smaller sized receptacle 100, which may have a volume of 30 mL, for example in the case of a patient with a persistent air leak. Such a smaller sized receptacle 100 may be carried in a pocket of the patient, or clipped to a belt of the patient, or clipped to a piece of clothing of the patient (FIG. 21). Alternatively the CDU connector 4 may be connected to a larger sized receptacle 101, which may have a volume of approximately 2 L. Such a larger sized receptacle 101 may be suitable for use with a bed-bound patient (FIG. 22).

The invention provides a pleural drain system 1 concerned with the drainage of air and fluid from the pleural lung space 60. The system 1 may be connected to a patient via a common pleural valve 3 in three configurations:
(1) Persistent Air Leak System
   Used for patients with a continuous air leak where fluid drainage is minimum. The system 1 contains the pleural valve 3, the quick disconnect connector 4 and the 30 mL chest drainage unit (CDU) 100 (FIG. 21).
(2) Ambulatory System
   Used for patients to drain both air and fluid, where patient is mobile. The system 1 contains the pleural valve 3, the quick disconnect connector 4 and the 500 mL chest drainage unit (CDU) 7 (FIG. 15).
(3) In Care System
   Used for patients to drain both air and fluid. Typically used where patient is in bed or is semi-mobile. The system 1 contains the pleural valve 3, the quick disconnect connector 4 and the 2000 mL chest drainage unit (CDU) 101 (FIG. 22). The CDU 101 can either be bed rail mounted or free-standing on the floor.

Referring to FIGS. 22(a) to 22(c) there is illustrated a receptacle 430 of another medical drainage device according to the invention, which is similar to the receptacle 7 of FIGS. 12 to 15, and similar elements in FIGS. 22(a) to 22(c) are assigned the same reference numbers.

In this case the receptacle 430 comprises a tube 431 located within the interior of the receptacle 430. The tube 431 extends from the inlet 50 to a region adjacent to a base 432 of the receptacle 430. The tube 431 acts as a fluid passageway for fluid that enters the receptacle 430 through the inlet 50.

The receptacle 430 reduces fluid foaming during collection, reduces noise and prevents fluid leaking from the collector 430 if the collector 430 is tipped over while the connector 4 is in place. The inner tube 431 provides a method of indication of air release due to bubbling in the collected fluid.

The inner tube 431 may be dipped in an anti-foam coating agent. By coating a base material with a material that reduces the tendency of the collected fluid to foam, the anti-foaming function may be achieved. This may be done by dipping a plastic tube, PVC/PU for example, into a coating solution and then leaving to dry.

When the collected fluid runs through the tube 431 this reduces foaming and noise of fluid dripping. FIGS. 22(b) and 22(c) illustrate the anti-reflux valve retainer 433. FIG. 22(c) illustrates the exploded collector cap assembly.

FIG. 22(d) illustrates another smaller sized receptacle 440 to which the CDU connector 4 may be connected, for example in the case of a patient with a persistent air leak. The receptacle 440 may have a volume of 30 mL. The receptacle 440 is substantially cylindrically shaped.

FIG. 22(e) illustrates another larger sized receptacle 441 to which the CDU connector 4 may be connected, for example in the case of a bed-bound patient. The receptacle 441 may have a volume of 2 L.

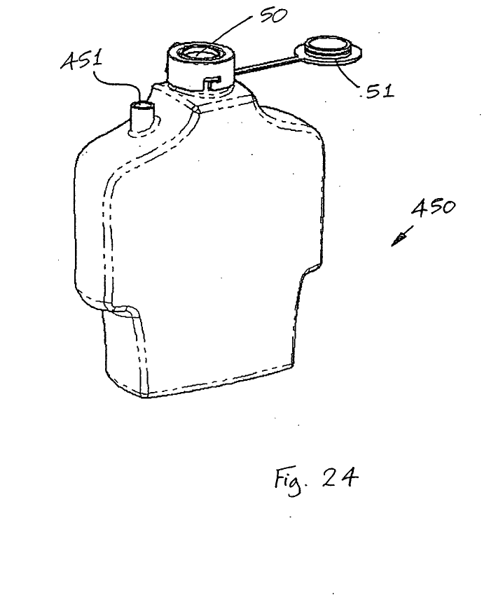

In FIG. 23 there is illustrated a further medical drainage device 110 according to the invention, which is similar to the device 1 of FIGS. 1 to 15, and similar elements in FIG. 23 are assigned the same reference numerals.

In this case the device 110 is suitable for use in urological applications to drain fluid from a bladder region of a patient. The CDU connector 4 may be connected to the receptacle 7 or to the larger sized receptacle 101 to suit the requirements of the patient.

An anti-bacterial filter may be located in the area of the shield openings 47 of the CDU connector 4. This would enable the system to be used in Urology applications.

Suction or vacuum may be applied to assist in drainage of the body fluid by adding a port to the body portion 40 of the CDU connector 4. This port may enable hospital vacuum supply to be connected into the pleural drainage system. Control of the applied suction or vacuum levels may be achieved by means of a fixed or variable regulator. Fixed suction may be achieved using a diaphragm seal. The diaphragm may crack open by a fixed amount regardless of the level of suction applied to the port. To achieve variable levels of suction the diaphragm opening may be adjustable. A mechanical turn knob may either compress or release the diaphragm depending on the level of suction required. A gauge on the supply side at the hospital outlet may signal the level of suction output.

FIG. 24 illustrates a receptacle 450 of another medical drainage device according to the invention, which is similar to the receptacle 7 of FIGS. 12 to 15, and similar elements in FIG. 24 are assigned the same reference numbers.

In this case the receptacle 450 comprises a vacuum port 45 1. The vacuum port 451 may be formed during manufacture of the receptacle 450, for example by moulding.

A valve may be provided at the vacuum port 451. The valve may be attached to the vacuum port 45 1, for example by bonding. The valve may be of any suitable valve type, for example a luer-lock valve.

A vacuum regulator may be provided at the vacuum port 451. The vacuum regulator may be a separate element from the receptacle 450, or alternatively may be integrated with the receptacle 450 to form a single unit.

Step down from 2000 mL to 500 mL to persistent air leak (PAL) system is easy using the device of the invention. Disconnection/reconnection of the collector 7 or the vent is possible without disturbing the patient's catheter/chest tube. The invention provides a complete self-seal system. Performance is unaffected by stability or orientation. No manual intervention is required to relieve positive pressure in the collector 7. The invention provides an out of the box ready to go system. No set up is required. The invention provides a lightweight, portable, easy to use and operate, comfortable to wear, and ergonomic design. The invention provides a dual anti-reflux dry valve system. No maintenance is required with the device of the invention.

Various specific materials have been outlined above as suitable for the component parts of the medical drainage device. It will be appreciated however that there are a variety of other suitable medical grade/compliant materials for the component parts of the medical drainage device.

The invention is not limited to the embodiments hereinbefore described, with reference to the accompanying drawings, which may be varied in construction and detail.

1. A device suitable for connection to a substantially tubular element, the device comprising:-
at least one substantially tubular member; and
one or more protrusions on a surface of the at least one tubular member;
the one or more protrusions being at least partially substantially deformable, upon engagement with a substantially tubular element, to sealingly engage the element.

2. A device as claimed in claim 1 wherein the protrusion is at least partially substantially compressible.

3. A device as claimed in claim 1 wherein the protrusion is radially deformable.

4. A device as claimed in claim 1 wherein the protrusion is mounted to the tubular member.

5. A device as claimed in claim 4 wherein the surface of the tubular member comprises a recess into which the protrusion is mounted.

6. A device as claimed in claim 4 wherein the protrusion is over-moulded to the tubular member.

7. A device as claimed in claim 1 wherein the protrusion is formed integrally with the tubular member.

8. A device as claimed in claim 1 wherein the protrusion is provided on an external surface of the tubular member.

9. A device as claimed in claim 8 wherein the protrusion upstands from the external surface of the tubular member.

10. A device as claimed in claim 1 wherein the tubular member is insertable at least partially into a substantially tubular element.

11. A device as claimed in claim 1 wherein the tubular member defines a lumen therethrough.

12. A device as claimed in claim 11 wherein the protrusion is provided on an internal surface of the tubular member.

13. A device as claimed in claim 11 wherein a substantially tubular element is insertable at least partially into the tubular member.

14. A device as claimed in claim 1 wherein the device comprises a plurality of protrusions spaced along the tubular member.

15. A device as claimed in claim 14 wherein the device comprises a first protrusion and a second protrusion, the second protrusion having a smaller radial dimension than the first protrusion.

16. A device as claimed in claim 15 wherein the second protrusion is closer to a free end of the tubular member than the first protrusion.

17. A device as claimed in claim 1 wherein the device comprises a first substantially tubular member and a second substantially tubular member.

18. A device as claimed in claim 17 wherein the longitudinal axis of the first tubular member is substantially aligned with the longitudinal axis of the second tubular member.

19. A device as claimed in claim 17 wherein the longitudinal axis of the first tubular member is substantially out of alignment with the longitudinal axis of the second tubular member.

20. A device as claimed in claim 17 wherein the device comprises a third substantially tubular member.

21. A device as claimed in claim 20 wherein the device is substantially “Y”-shaped.

22. A device as claimed in claim 1 wherein the protrusion extends circumferentially around the tubular member.

23. A device as claimed in claim 22 wherein the protrusion is substantially annular in shape.

24. A device as claimed in claim 1 wherein the tubular member is substantially cylindrical in shape.

25. A device as claimed in claim 1 wherein the tubular member is substantially rigid.

26. A device as claimed in claim 1 wherein the protrusion is of a first material and the tubular member is of a second material different to the first material.

27. A device as claimed in claim 26 wherein the first material is more deformable than the second material.

28. A device as claimed in claim 27 wherein the first material is more flexible than the second material.

29. A medical device as claimed in claim 1.

30. A medical drainage device as claimed in claim 29.
